SEQUENCE LISTING (63 sequences)

SEQ ID NO: 1 - DNA, 20 bp, synthetic construct
  source 1..20: mol_type = other DNA
gcttccaggccatattggag

SEQ ID NO: 2 - DNA, 19 bp, synthetic construct
  source 1..19: mol_type = other DNA
gggggcatgacctcatctt

SEQ ID NO: 3 - DNA, 21 bp, synthetic construct
  source 1..21: mol_type = other DNA
tgctgagatggtgacctacga

SEQ ID NO: 4 - DNA, 21 bp, synthetic construct
  source 1..21: mol_type = other DNA
ccaaaggcagagacaaagtga

SEQ ID NO: 5 - DNA, 20 bp, synthetic construct
  source 1..20: mol_type = other DNA
gcgttctgggtaccatccta

SEQ ID NO: 6 - DNA, 20 bp, synthetic construct
  source 1..20: mol_type = other DNA
gctctgagcccttggtgtag

SEQ ID NO: 7 - DNA, 23 bp, synthetic construct
  source 1..23: mol_type = other DNA
agggtttagttttgagttgacgg

SEQ ID NO: 8 - DNA, 21 bp, synthetic construct
  source 1..21: mol_type = other DNA
ccccgcttttgtcatattccg

SEQ ID NO: 9 - DNA, 20 bp, synthetic construct
  source 1..20: mol_type = other DNA
catactcgcgggaaagacca

SEQ ID NO: 10 - DNA, 20 bp, synthetic construct
  source 1..20: mol_type = other DNA
cgtcttctcggccatacaca

SEQ ID NO: 11 - DNA, 19 bp, synthetic construct
  source 1..19: mol_type = other DNA
tctaggcaatgccgttcac

SEQ ID NO: 12 - DNA, 19 bp, synthetic construct
  source 1..19: mol_type = other DNA
gagcacatgggcaccatac

SEQ ID NO: 13 - DNA, 20 bp, synthetic construct
  source 1..20: mol_type = other DNA
agacccaggtcagcatcttg

SEQ ID NO: 14 - DNA, 21 bp, synthetic construct
  source 1..21: mol_type = other DNA
gccgattctttgttcccttga

SEQ ID NO: 15 - DNA, 20 bp, synthetic construct
  source 1..20: mol_type = other DNA
ggatccgagcatcttatcca

SEQ ID NO: 16 - DNA, 20 bp, synthetic construct
  source 1..20: mol_type = other DNA
ggtggtactcccgctgtaaa

SEQ ID NO: 17 - DNA, 20 bp, synthetic construct
  source 1..20: mol_type = other DNA
gtgtccccactgatgaggag

SEQ ID NO: 18 - DNA, 20 bp, synthetic construct
  source 1..20: mol_type = other DNA
tatgggtccagtcccttctg

SEQ ID NO: 19 - DNA, 20 bp, synthetic construct
  source 1..20: mol_type = other DNA
attccttcatgtcggacgag

SEQ ID NO: 20 - DNA, 20 bp, synthetic construct
  source 1..20: mol_type = other DNA
actgagaagccccctcaaat

SEQ ID NO: 21 - DNA, 20 bp, synthetic construct
  source 1..20: mol_type = other DNA
cccggcagagacggttaaaa

SEQ ID NO: 22 - DNA, 20 bp, synthetic construct
  source 1..20: mol_type = other DNA
tccctgagccgaatcatcct

SEQ ID NO: 23 - DNA, 20 bp, synthetic construct
  source 1..20: mol_type = other DNA
ccaggcttctcctcactgtc

SEQ ID NO: 24 - DNA, 20 bp, synthetic construct
  source 1..20: mol_type = other DNA
gccccctcttcatctaggac

SEQ ID NO: 25 - DNA, 20 bp, synthetic construct
  source 1..20: mol_type = other DNA
gtaaatctgcgggatgatgg

SEQ ID NO: 26 - DNA, 20 bp, synthetic construct
  source 1..20: mol_type = other DNA
agcagggtcaaaatcgtctg

SEQ ID NO: 27 - DNA, 20 bp, synthetic construct
  source 1..20: mol_type = other DNA
agaactgtgtacggagcgtg

SEQ ID NO: 28 - DNA, 20 bp, synthetic construct
  source 1..20: mol_type = other DNA
gtgcaccatctttggcagtg

SEQ ID NO: 29 - DNA, 22 bp, synthetic construct
  source 1..22: mol_type = other DNA
ggatgtacctgactcagagggt

SEQ ID NO: 30 - DNA, 22 bp, synthetic construct
  source 1..22: mol_type = other DNA
tacctgcacagttgacagccac

SEQ ID NO: 31 - DNA, 23 bp, synthetic construct
  source 1..23: mol_type = other DNA
catagcaacagagtgccttccac

SEQ ID NO: 32 - DNA, 22 bp, synthetic construct
  source 1..22: mol_type = other DNA
cctgggaacaaacttgagagcc

SEQ ID NO: 33 - DNA, 22 bp, synthetic construct
  source 1..22: mol_type = other DNA
acgatggctcatctctcaaggc

SEQ ID NO: 34 - DNA, 22 bp, synthetic construct
  source 1..22: mol_type = other DNA
tgtggtgcaactgaggcttcgt

SEQ ID NO: 35 - DNA, 22 bp, synthetic construct
  source 1..22: mol_type = other DNA
agtcactggtgctaacacaggc

SEQ ID NO: 36 - DNA, 22 bp, synthetic construct
  source 1..22: mol_type = other DNA
tgttccctgtgacggcttggat

SEQ ID NO: 37 - DNA, 22 bp, synthetic construct
  source 1..22: mol_type = other DNA
cctggtcaacaatgctggcatc

SEQ ID NO: 38 - DNA, 22 bp, synthetic construct
  source 1..22: mol_type = other DNA
catgctcagagtcacctcgatc

SEQ ID NO: 39 - DNA, 23 bp, synthetic construct
  source 1..23: mol_type = other DNA
gctttctgccaagaactgctaca

SEQ ID NO: 40 - DNA, 23 bp, synthetic construct
  source 1..23: mol_type = other DNA
ccagatggttcactccaaactgc

SEQ ID NO: 41 - DNA, 23 bp, synthetic construct
  source 1..23: mol_type = other DNA
ggacaggattgacagattgatag

SEQ ID NO: 42 - DNA, 20 bp, synthetic construct
  source 1..20: mol_type = other DNA
atcgctccaccaactaagaa

SEQ ID NO: 43 - DNA, 19 bp, synthetic construct
  source 1..19: mol_type = other DNA
ggcttcgggactggtagcc

SEQ ID NO: 44 - DNA, 20 bp, synthetic construct
  source 1..20: mol_type = other DNA
gcggcttgatatcctcagtg

SEQ ID NO: 45 - DNA, 24 bp, synthetic construct
  source 1..24: mol_type = other DNA
accaggatttgtgtggtggtcaga

SEQ ID NO: 46 - DNA, 24 bp, synthetic construct
  source 1..24: mol_type = other DNA
agccattgctgaggtcacacagta

SEQ ID NO: 47 - DNA, 24 bp, synthetic construct
  source 1..24: mol_type = other DNA
ccatatgaatgcggattcgaccct

SEQ ID NO: 48 - DNA, 26 bp, synthetic construct
  source 1..26: mol_type = other DNA
gctcatggtagtggaagtagaagagc

SEQ ID NO: 49 - DNA, 21 bp, synthetic construct
  source 1..21: mol_type = other DNA
ggaagaccactcgcattcctt

SEQ ID NO: 50 - DNA, 22 bp, synthetic construct
  source 1..22: mol_type = other DNA
tcgcactttggtattcttggag

SEQ ID NO: 51 - DNA, 21 bp, synthetic construct
  source 1..21: mol_type = other DNA
gctaacccaagacaaccaacc

SEQ ID NO: 52 - DNA, 21 bp, synthetic construct
  source 1..21: mol_type = other DNA
cagactcctagaagggaccca

SEQ ID NO: 53 - DNA, 19 bp, synthetic construct
  source 1..19: mol_type = other DNA
gcgggagtaccaccataca

SEQ ID NO: 54 - DNA, 22 bp, synthetic construct
  source 1..22: mol_type = other DNA
gatgatggcaagggtgatagga

SEQ ID NO: 55 - DNA, 25 bp, synthetic construct
  source 1..25: mol_type = other DNA
gtctgacttttcctatacgtctcca

SEQ ID NO: 56 - DNA, 20 bp, synthetic construct
  source 1..20: mol_type = other DNA
tgggggagtcagaatgcaac

SEQ ID NO: 57 - DNA, 21 bp, synthetic construct
  source 1..21: mol_type = other DNA
acacattcgaggaaccaacct

SEQ ID NO: 58 - DNA, 20 bp, synthetic construct
  source 1..20: mol_type = other DNA
ggggatgtggcgtcttgtag

SEQ ID NO: 59 - DNA, 25 bp, synthetic construct
  source 1..25: mol_type = other DNA
gtgcttggaataatcatagtgtagc

SEQ ID NO: 60 - DNA, 23 bp, synthetic construct
  source 1..23: mol_type = other DNA
gggctaggattagttcagagtgt

SEQ ID NO: 61 - DNA, 28 bp, synthetic construct
  source 1..28: mol_type = other DNA
atctagcccagaatttgttgttccagtg

SEQ ID NO: 62 - DNA, 19 bp, synthetic construct
  source 1..19: mol_type = other DNA
tctaggccagagggcgcag

SEQ ID NO: 63 - DNA, 19 bp, synthetic construct
  source 1..19: mol_type = other DNA
saaggtcanntsaaggtca

(19) United States
(12) Patent Application Publication
(10) Pub. No.: US 2024/0216412 A1
(43) Pub. Date: Jul. 4, 2024

(54) OXIDIZED NICOTINAMIDE ADENINE DINUCLEOTIDE (NAD+) PRECURSOR TREATMENT FOR HEART FAILURE ALLEVIATION INDEPENDENT OF MITOCHONDRIAL PROTEIN DEACETYLATION

(71) Applicant: University of Washington, Seattle, WA (US)

(72) Inventors: RONG TIAN, Seattle, WA (US); MATTHEW WALKER, Seattle, WA (US)

(73) Assignee: University of Washington, Seattle, WA (US)

(21) Appl. No.: 18/517,421

(22) Filed: Nov. 22, 2023

Related U.S. Application Data
(60) Provisional application No. 63/427,449, filed on Nov. 22, 2022.

(51) Int. Cl. A61K 31/706; A61P 9/00

(52) U.S. Cl. CPC A61K 31/706; A61P 9/00

(57) ABSTRACT

The current disclosure provides provides boosting intracellular NAD⁺ with nicotinamide riboside chloride (NR) to protect from cardiac dysfunction, NR being a natural NAD⁺ precursor that bypasses nicotinamide phosphoribosyltransferase (NAMPT) to stimulate intracellular NAD⁺ synthesis.

### CROSS-REFERENCE TO RELATED APPLICATION

This application claims the benefit of U.S. Provisional Patent Application No. 63/427,449 filed on Nov. 22, 2022, the contents of which are incorporated herein by reference in its entirety as if fully set forth herein.

### STATEMENT REGARDING FEDERALLY SPONSORED RESEARCH OR DEVELOPMENT

This invention was made with government support under Grant No. HL-110349, awarded by the National Institutes of Health (NIH). The government has certain rights in the invention.

### REFERENCE TO SEQUENCE LISTING

The present application is being filed along with a Sequence Listing in electronic format. The Sequence Listing is provided as a file entitled W149-0039US.xml created on Mar. 8, 2024, which is 55.2 kilobytes in size. The information in the electronic format of the Sequence Listing is incorporated herein by reference in its entirety.

### FIELD OF THE DISCLOSURE

The current disclosure provides use of an oxidized nicotinamide adenine dinucleotide (NAD⁺) precursor treatment to alleviate heart failure in subjects with preexisting heart failure.

### BACKGROUND OF THE DISCLOSURE

Heart failure is a devastating disease with unacceptable mortality rate despite rigorous medical treatment (Roth et al., 2015). Cardiac myocytes have the highest content of mitochondria and mitochondrial dysfunction is an important mechanism of heart failure (Lopaschuk et al., 2021; Zhou and Tian, 2018). Numerous studies have proposed mitochondrial dysfunction to promote heart failure (Dorn et al., 2015; Ingwall, 2009; Karamanlidis et al., 2013; Neubauer, 2007; Oka et al., 2020; Zhou and Tian, 2018). Drastic shifts in mitochondrial structure, biogenesis, and fuel utilization are well-documented in failing heart and yet, no therapies exist to treat mitochondrial dysfunction in diseased heart (Dorn et al., 2015; Lopaschuk et al., 2021; Zhou and Tian, 2018). An important component of the mitochondrial crisis is the transcriptional downregulation of nuclear receptor pathways mediated by PGC-1α (peroxisome proliferator-activated receptor gamma coactivator 1α), PPARα (proliferator-activated receptor α), and the RXRα (retinoid X receptor) signaling complexes (Lai et al., 2014; Lehman and Kelly, 2002; Lopaschuk et al., 2010). Suppression of these pathways leads to reduced expression and activity of the fatty acid oxidation (FAO) machinery and components of the electron transport chain (ETC) (Bugger et al., 2010; Karamanlidis et al., 2010; Lopaschuk et al., 2010). These changes reduce the capacity for FA β-oxidation in mitochondria and limit ATP production via oxidative phosphorylation (OXPHOS) (Lopaschuk et al., 2010; Zhou and Tian, 2018).

Mitochondrial dysfunction not only disrupts the supply and transfer of adenosine triphosphate (ATP) but also impairs nicotinamide adenine dinucleotide (NAD⁺) recycling. Electrons extracted from substrate catabolism are carried as NADH which is oxidized at complex I in the mitochondrial electron transport chain (ETC) to regenerate NAD⁺ (Karamanlidis et al., 2013). Thus, mitochondria are critical regulators of the redox balance between oxidized (NAD⁺) and reduced (NADH) forms of cellular NAD. In turn, the NAD redox balance, i.e. NAD⁺/NADH ratio, critically regulates enzymatic reactions in cell metabolism. A change in NAD⁺/NADH ratio also affects the availability of NAD⁺ which functions as a co-substrate for sirtuin deacylase, ADP-ribose transferases, and cyclic ADP-ribose synthases that post-translationally modify proteins while consuming NAD⁺ and producing nicotinamide (NAM) (Houtkooper et al., 2010). Imbalanced NADH production and oxidation in mitochondria can be a positive regulator of pathological remodeling in failing hearts (Karamanlidis et al., 2013; Lee et al., 2016). In support of this, multiple models of cardiac stress show depressed levels of NAD⁺ and a decline in enzymes responsible for the biosynthesis and salvage of NAD⁺ (Byun et al., 2019; Chiao et al., 2021; Dierickx et al., 2022; Diguet et al., 2018; Lee et al., 2016; Oka et al., 2021; Yamamoto et al., 2014). Nicotinamide phophoribosyltransferase (NAMPT) levels decrease in heart failure compromising the ability in the salvage of nicotinamide (NAM) back into NAD⁺ (Diguet et al., 2018). Normalizing the redox imbalance by raising NAD⁺ levels genetically or pharmacologically has been shown an effective preventive strategy in suppressing cardiac hypertrophy and improving cardiac function in multiple animal models of heart failure (Diguet et al., 2018; Lee et al., 2016; Martin et al., 2017).

Despite the well-documented beneficial effects of raising NAD⁺ levels in failing hearts, understanding of the underlying mechanisms remains very limited. Most evidence suggests mechanisms involving activation of Sirt3, the primary mitochondrial protein deacetylase, that alleviates mitochondrial protein hyperacetylation (Karamanlidis et al., 2013; Lee et al., 2016; Martin et al., 2017; Tong et al., 2021; Zhang et al., 2018). One study showed the protection afforded by increasing NAD⁺ in a mitochondrial mutation induced cardiomyopathy was dependent on Sirt3 (Martin et al., 2017). This notion, however, is recently challenged by a report showing extreme acetylation of mitochondrial protein in a double mutant mouse model does not promote heart failure (Davidson et al., 2020).

Accordingly, before the present disclosure, strategies to increase cellular NAD⁺ level have shown protection in multiple models of heart failure, but molecular mechanisms remain unclear.

### SUMMARY OF THE DISCLOSURE

The present disclosure provides methods and compositions for treatment with a NAD⁺ precursor in subjects with established cardiac dysfunction due to chronic pressure overload, improving mitochondrial function and blunting the progression of the disease. Furthermore, the benefit is observed regardless of Sirt3-mediated mitochondrial protein deacetylation, a widely held mechanism for NAD⁺ based therapy for heart failure. Boosting NAD⁺ level improves the function of NAD(H) redox sensitive short-chain dehydrogenase/reductases (SDR) family proteins. Upregulation of Mrpp2, a multifunctional SDR protein and a key component of mitochondrial ribonuclease P, improves mtDNA transcripts processing and electron transport chain function. Activation of SDRs in retinol metabolism pathway stimulates RXRα/PPARα signaling and restores fatty acid oxidation. These findings identify SDR proteins as regulators of mitochondrial function and molecular targets of NAD⁺ based therapy.

Increasing NAD⁺ levels in failing hearts improves mitochondrial function and protects against the development of heart failure through mechanisms dependent of Sirt3-mediated protein deacetylation. Upon inducing cardiac dysfunction by pressure overload in SIRT3 deficient (KO) mice, their response was compared to nicotinamide riboside chloride (NR) treatment with wild-type (WT) mice subjected to the same procedures. NR is a NAD precursor that enters NAD⁺ salvage pathway through nicotinamide riboside kinase (Nrk), and it has been shown to increase intracellular NAD⁺ level independent of NAMPT (Diguet et al., 2018). The present disclosure illustrates that increasing NAD⁺ level protected both KO and WT mice from the progression of heart failure, and novel mechanisms through which the treatment improves mitochondrial function in failing hearts.

### BRIEF DESCRIPTION OF THE DRAWINGS

FIGS. 1A-1J: Increasing NAD⁺ level blunted the development of heart failure in Sirt3 deficient mice. (1A) Experimental protocol. At 4 weeks post-transverse aortic constriction (TAC) mice were treated with nicotinamide riboside chloride (NR) or vehicle (saline) for 8 weeks. (1B) Ratio of heart weight/tibia length (HW/TL) was measured after 8 weeks of treatment. (1C-1D) Representative echocardiograms at different time points after TAC for WT and Sirt3Ko mice. (1E-1F) Fractional shortening (FS) for WT and Sirt3Ko mice quantified by echocardiography. (1G) Left ventricular (LV) dilation (left ventricular internal diameter at end diastole, LVID; d) quantified by echocardiography for WT and Sirt3Ko mice. (1H) Representative LV cross sections of Sirt3Ko TAC+/−NR stained with Masson's trichrome. Scale bars, top 1 mm and bottom 100 μM. (1I) Quantification of fibrotic area. (1J) Lung edema (wet/dry lung weight) was measured after 8 weeks of treatment. All data presented as mean±SEM. P value calculated by mixed-effects analysis followed by Tukey post hoc analysis (1E and 1F) or two-way ANOVA followed by Tukey post hoc analysis (1B, 1G, 1I, and 1J) (n=at least 5 per group except I n=3, *P<0.05 vs WT sham veh, $P<0.05 vs Sirt3Ko sham veh, and #P<0.05 vs TAC veh).

FIGS. 2A-2G: NR treatment restored NAD(H) redox but not mitochondrial protein hyperacetylation in Sirt3Ko hearts. (2A) Sirtuin deacetylase reactions require NAD⁺. NAD⁺ level is determined by the rate of NAD⁺ synthesis in the Salvage Pathway and by the NAD⁺/NADH ratio maintained by mitochondrial function. Nicotinamide Phosphoribosyltransferase (NAMPT) catalyzes the conversion of nicotinamide (NAM) into nicotinamide mononucleotide (NMN). NAM not salvaged into NAD⁺ is converted to methyl nicotinamide (MeNAM). Nicotinamide riboside kinase (Nrk) catalyzes the first step of NR to NAD⁺ bypassing NAMPT. (2B) Cardiac NAD⁺ and NADH levels determined by enzymatic cycling assay and (2C) NAM and MeNAM determined by LCMS-based metabolomics in Sirt3Ko subjected to sham or TAC surgery+/−NR treatment and from (2D-2E) WT mice. (2F) Tissue NAD⁺/NADH ratio. (2G) Global acetylation levels of isolated cardiac mitochondria by Western blotting (top) and Ponceau s staining used for loading control (bottom). All data presented as mean±SEM. P value calculated by two-way ANOVA followed by Tukey post hoc analysis (n=at least 5 per group except G n=3, *P<0.05 vs WT sham veh, $P<0.05 vs Sirt3Ko sham veh, and #P<0.05 vs TAC veh).

FIGS. 3A-3G: Increasing intracellular NAD⁺ level prevents metabolic reprogramming after TAC. (3A) RNA sequencing protocols were performed on 4 WT hearts per group. Top 10 up- and down-regulated pathways in failing mouse hearts +/−NR treatments (TAC NR versus TAC Veh) identified in the Gene Set Enrichment Analysis (GSEA) analysis (false discovery rate <0.05, p<0.05). (3B) Representative gene set enrichment plot of the top upregulated pathway in NR treated TAC heart vs TAC vehicle. The leading edge subset highlights gene members that contribute most to the enrichment score. In NR treated failing hearts, the leading edge subset members are enriched in mitochondrial (MT-) DNA encoded transcripts. (3C-3D) Expression level of mitochondrial DNA (MT) encoded and nuclear encoded electron transport chain (ETC) transcripts determined by real-time polymerase chain reaction (rt-PCR) from Sirt3Ko heart and WT hearts. (3E-3F) Electron transport chain (ETC) enzymatic activities: Complex IV (COIV), Complex I (CI) and Citrate Synthase (CS) in Sirt3Ko heart lysate and WT heart lysate. (3G) Representative Oxygen consumption tracing and rates of cardiac mitochondria supported with Complex I carbon substrates. All data presented as mean±SEM. Dashed line indicates sham vehicle. P value calculated by one-way or two-way ANOVA followed by Tukey post hoc analysis (n=at least 5 per group, *P<0.05 vs WT sham veh, $P<0.05 vs Sirt3Ko sham veh, and #P<0.05 vs TAC veh).

FIGS. 4A-4K: RNAse P complex activity is impaired in failing hearts. (4A) Mitochondrial RNA junctions were measured in total heart RNA from WT and Sirt3Ko Sham and TAC hearts normalized to 18s rRNA. (4B) Expression levels of the short chain dehydrogenase (SDR) Mrpp2 in WT and Sirt3Ko mouse hearts. (4C) Mrpp2 dehydrogenase activity. (4D) Representative immunoblot of Mrpp2 and ATPβ as loading control (4E) Quantification of densitometry from Western blotting subunits of RNAse P complex (mrpp1 and mrpp2) in the WT TAC hearts +/−NR. (4F) Representative immunoblot of Mrpp2 in Sirt3Ko hearts +/−NR and Ponceau s staining for loading control (4G) Quantification of densitometry normalized to total protein per lane. (4H) Representative immunoblot showing decreased protein levels of mitochondrial DNA (mtDNA) encoded Complex IV subunit I (Cox1) (antibody mt-Col) and Ponceau s staining for loading control. (4I) Quantification of densitometry from Western blotting mtDNA-encoded Complex I (Nd-1) and Cox1 (antibody mt-Col). (4J) Quantification of short chain acylcarnitines (ACs) demonstrating elevated C5 and C5-OH carnitines in failing heart tissue from WT and from (4K) Sirt3Ko. All data presented as mean±SEM. Dashed line indicates sham vehicle. P value calculated by one-way or two-way ANOVA followed by Tukey post hoc analysis (n=at least 5 per group, *P<0.05 vs WT sham veh, $P<0.05 vs Sirt3Ko sham veh, and #P<0.05 vs TAC Veh).

FIGS. 5A-5G: Activation of NAD(H) redox sensitive short-chain dehydrogenase/reductase (SDR) proteins in failing hearts removed the bottleneck of retinol oxidation. (5A) Pathway analysis showed that SDRs in murine heart are significantly enriched in metabolic pathways involving retinol metabolism and steroid biosynthesis. 43 SDR genes were found in our RNA sequencing data. Genes annotated to be in retinol metabolism shaded in green. All other SDRs shaded in blue. (5B) Retinoic acid biosynthesis pathway. NAD(P)(H) dependent SDRs catalyze the rate-limiting step in the oxidation of retinol into retinoic acid. NR stimulates Nicotinamide riboside kinase (Nrk) to increase NAD⁺ level to stimulate SDRs in failing hearts. (5C-5F) Quantification of retinol and retinyl ester in WT and Sirt3Ko hearts subjected to sham and TAC+/−NR treatments. (5G) Correlation curve between the intracellular NAD⁺/NADH ratio and retinol concentration (pmol/g) in all groups. All data presented as mean±SEM. P value calculated by two-way ANOVA followed by Tukey post hoc analysis n=at least 5 per group, *P<0.05 vs sham veh, #P<0.05 vs TAC veh.

FIGS. 6A-6G: Raising NAD⁺ level restored PPARα/RXRα signaling in failing hearts and improved fatty acid beta oxidation. (6A) Genes from upregulated pathways in the GSEA analysis (defined by a family-wise error rate [FWER]<0.25) were further analyzed using iRegulon (Janky et al., 2014). TF's/Motifs identified are clustered by similarity and ranked by normalized enrichment score (NES). RXRα was one of the top 3 TF's identified among the upregulated genes in NR vs vehicle treated TAC hearts. The highest NES scores were motifs containing the consensus sequence AGGTCA which are known binding sites for RXRα and its interaction partner PPARα. The sequence illustrated in FIG. 6A is SAAGGTCANNTSAAGGTCA (SEQ ID NO: 63). (6B-6C) Expression level of transcription factors determined by real-time polymerase chain reaction (rt-PCR) from WT and Sirt3Ko hearts. (6D-6E) Expression levels of proliferator-activated receptor α (Pparα) target genes in WT and Sirt3Ko hearts. (6F-6G) Levels of acylcarnitine species from WT and Sirt3Ko hearts. Acyl chain lengths are denoted by the numbers. All data presented as mean±SEM. Dashed line indicates sham vehicle. P value calculated by one-way or two-way ANOVA followed by Tukey post hoc analysis (n=at least 5 per group, *P<0.05 vs sham veh, $P<0.05 vs Sirt3Ko sham veh, and #P<0.05 vs TAC-Veh).

FIGS. 7A-7H: Activation of PPARα signaling was required for the benefit of raising NAD⁺ in TAC hearts. (7A-7B) Fractional shortening (FS) % from Sirt3Ko and WT mice subjected to isoproterenol (ISO) induced (mini-pump 14 days) heart failure +/−NR (500 mg/kg body weight per day) treatments. A subgroup of NR treated mice were administered the Pparα/RXR inhibitor GW6471 daily. (7C) Cardiac hypertrophy (heart weight/tibia length, HW/TL) assessment in the WT mice. (7D) Fatty acid supported (Palmitoylcarnitine) oxygen consumption rate in isolated mitochondria from each group of treated WT mice. (7E) State 3 adenosine diphosphate (ADP) supported respiration was improved with NR treatments and blocked with GW6471 administration. (7F) NR in the presence of GW6471 failed to preserve PPARα/RXRα target gene expression in ISO treated hearts. (7G-7H) Chromatin immunoprecipitation (ChIP) assay to assess binding occupancy of RXRα to the PPARα response element (PPRE) in the Acadm promoter in NR treated WT and Sirt3Ko ISO mouse hearts +/−the selective inhibitor GW6471 All data presented as mean±SEM. Dashed line indicates sham vehicle. P value calculated by one-way or two-way ANOVA followed by Tukey post hoc analysis (n=at least 5 per group, *P<0.05 vs Day 0 saline A-B or Day 14 saline C, #P<0.05 vs Day 14 ISO+Veh).

FIGS. 8A-8E: Increasing NAD⁺ level blunted the development of heart failure in Sirt3 deficient mice. (8A) Immunoblotting confirming the deletion of Sirtuin 3 (Sirt3) and mitochondrial hyperacetylation in mouse heart. (8B-8C) Survival curve analysis of WT and Sirt3ko. (8D) Expression levels of nppa and ctgf. (8E) Heatmap of metabolites from WT and Sirt3Ko heart 12 weeks after TAC+/−NR. All data presented as mean±SEM. Dashed line indicates sham vehicle values. P value calculated by two-way ANOVA followed by Tukey post hoc analysis (n=at least 5 per group), *P<0.05 vs WT Sham Veh, #P<0.05 vs TAC Veh).

FIGS. 9A-9B: RNAse P complex activity is impaired in failing hearts. (9A) Mrpp1 expression levels measured by real-time PCR. (9B) Quantification of densitometry from Western blotting mtDNA-encoded Complex I (Nd-1) and mt-Col in Sirt3Ko hearts. All data presented as mean±SEM. P value calculated by two-way ANOVA followed by Tukey post hoc analysis (n=at least 5 per group, *P<0.05 vs sham veh, #P<0.05 vs TAC Veh).

FIGS. 10A-10B: Activation of NAD(H) redox sensitive short-chain dehydrogenase/reductase (SDR) proteins in failing hearts removed the bottleneck of retinol oxidation. (10A) Gene Ontology in the Database for Annotation, Visualization and Integrated Discovery (DAVID). (10B) Expression levels of Rdh transcripts in WT and Sirt3Ko hearts 12 weeks after TAC+/−NR. All data presented as mean±SEM. P value calculated by two-way ANOVA followed by Tukey post hoc analysis (n=at least 5 per group, *P<0.05 sham vehicle, #P<0.05 vs TAC veh).

FIG. 11: Raising NAD⁺ level restored PPARα/RXRα signaling in failing hearts and improved fatty acid beta oxidation. Heatmap of acylcarnitine species from WT and Sirt3Ko hearts 12 weeks after TAC+/−NR treatments.

FIGS. 12A-12C: Activation of PPARα signaling was required for the benefit of raising NAD⁺ in TAC hearts. (12A) Cardiac hypertrophy (heart weight/tibia length, HW/TL) assessment in the Sirt3KO mice. (12B) State 3 adenosine diphosphate (ADP) supported respiration was improved with NR treatments and blocked with GW6471 administration in Sirt3Ko mice. (12C) NR in the presence of GW6471 failed to preserve PPARα/RXRα target gene expression in ISO treated Sirt3Ko hearts. All data presented as mean±SEM. Dashed line indicates sham vehicle values. P value calculated by two-way ANOVA followed by Tukey post hoc analysis (n=at least 5 per group), *P<0.05 saline veh, #P<0.05 vs ISO veh).

FIGS. 13A-13F: NR treatment increased NAD⁺ levels in hearts and livers, with greater increases exhibited by IP injection administration compared to oral administration. (2A) Cardiac tissue NAD⁺ levels determined by enzymatic cycling assay after 0 days; after 3 or 7 days oral NR administration; and after 10 days NR administration by IP injection. (2B) Cardiac tissue NADH levels. (2C) Cardiac tissue NAD⁺/NADH ratio. (2D) Liver tissue NAD⁺ levels determined by enzymatic cycling assay after 0 days; after 3 or 7 days oral NR administration; and after 10 days NR administration by IP injection. (2E) Liver tissue NADH levels. (2F) Liver tissue NAD⁺/NADH ratio.

### DETAILED DESCRIPTION

In recent years, much attention has been focused on NAD⁺-dependent deacetylase, sirtuins, as mediators of heart disease (Hershberger et al., 2017; Karamanlidis et al., 2013; Koentges et al., 2015; Lee et al., 2016; Martin et al., 2017; Zhang et al., 2018). Loss of NAD⁺ homeostasis has been reported to negatively impact sirtuin function and directly contribute to the development of heart failure. Mitochondrial localized Sirt3 is known to be repressed in several models of heart failure and is associated with poor outcomes (Horton et al., 2016; Koentges et al., 2015; Lee et al., 2016; Martin et al., 2017; Sundaresan et al., 2009; Zhang et al., 2018). Restoring NAD⁺ level has been reported to re-activate Sirt3 and alleviate heart failure in mice (Lee et al., 2016; Martin et al., 2017). However, a study found that extreme mitochondrial protein acetylation did not exacerbate heart failure in mice thus raising questions regarding the mechanistic role of sirtuin mediated protein deacetylation in the development of heart failure (Davidson et al., 2020).

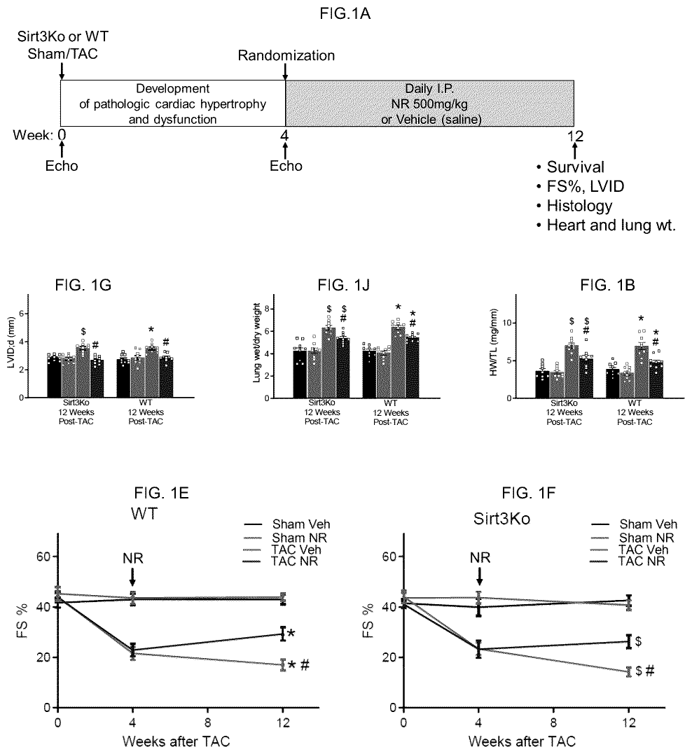

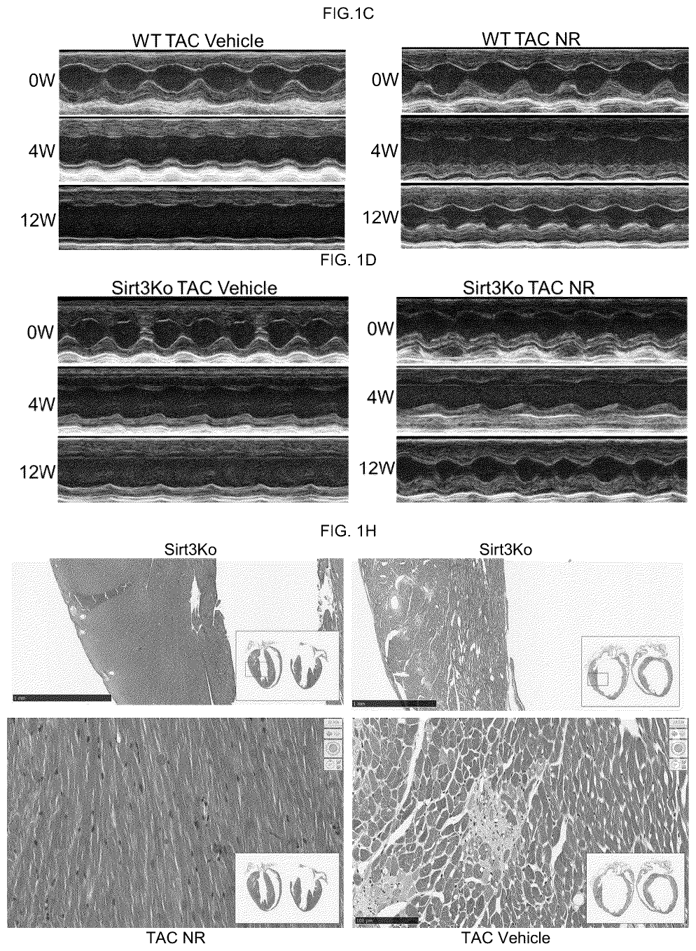

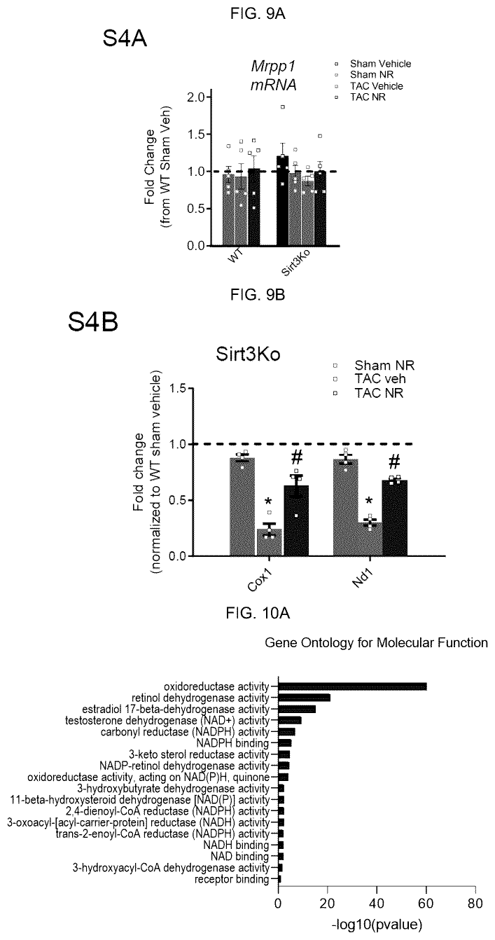

The present disclosure provides boosting intracellular NAD⁺ with nicotinamide riboside chloride (NR) to protect Sirtuin 3 (Sirt3) knockout mice from heart failure. NR is a natural NAD precursor that bypasses nicotinamide phosphoribosyltransferase (NAMPT) to stimulate intracellular NAD⁺ synthesis via the Nrk reaction (Bieganowski and Brenner, 2004; Canto et al., 2012; Diguet et al., 2018; Trammell et al., 2016). NR treatment is shown to be protective independent of mitochondrial protein deacetylation, by demonstrating NR's efficacy in Sirtuin 3 (Sirt3) deficient mice subjected to chronic pressure overload induced heart failure. Immunoblotting confirmed the absence of Sirt3 and increased lysine acetylation in mitochondrial extracts from the knockout hearts (FIG. 9A). Pressure overload was induced by transverse aortic constriction (TAC) surgery for 12 weeks. Intracellular NAD⁺ level was increased when mice had already developed cardiac hypertrophy and dysfunction, to model a therapeutic approach (FIG. 1A). After 4 weeks of chronic pressure overload, cardiac function was assessed using trans-thoracic M-mode echocardiography.

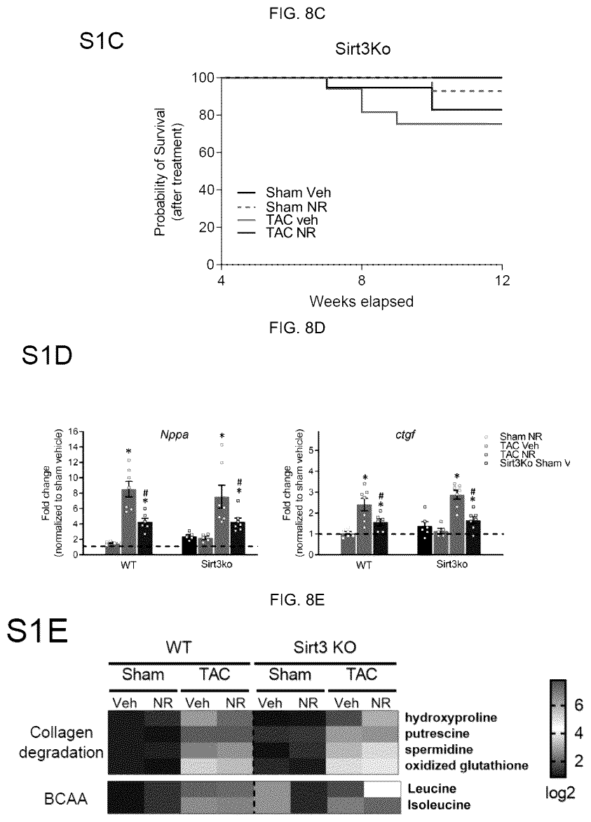

Mice randomly received either NR (500 mg/kg body weight) (Canto et al., 2012) or vehicle saline daily for eight-weeks. Representative M-mode images for the echocardiography can be found in FIGS. 1C and 1D. Four weeks of chronic pressure overload caused similar declines in left ventricular fractional shortening (FS %) in mice randomized to NR or vehicle groups for either WT or Sirt3Ko (FIGS. 1E and 1F). Kaplan-Meier survival curve analysis showed no significant difference in mortality between mice that received NR or vehicle for eight weeks (FIGS. 8B and 8C). NR treatment preserved FS % in WT TAO mice during the 8-week treatment (FIG. 1E). The treatment also protected the Sirt3Ko TAO mice from advances of heart failure as compared with mice that received the vehicle treatment (FIGS. 1B-1J). Similar to NR treated WT, cardiac hypertrophy, left ventricular cavity dilation, and lung edema were less severe in Sirt3Ko TAO mice that received NR treatment (FIGS. 1B-1J). All echocardiography parameters can be found in Table 1 below.

| | Wildtype | | | | Sirt3Ko | | | |
|---|---|---|---|---|---|---|---|---|
| | Sham | Sham | TAC | TAC | Sham | Sham | TAC | TAC |
| | Veh | NR | Veh | NR | Veh | NR | Veh | NR |
| IVS; d | 1.08 ± | 1.12 ± | 1.15 ± | 1.30 ± | 1.18 ± | 1.04 ± | 1.28 ± | 1.22 ± |
| (mm) | 0.07 | 0.03 | 0.10 | 0.07 | 0.04 | 0.06 | 0.05 | 0.07 |
| IVS; s | 1.62 ± | 1.72 ± | 1.57 ± | 1.78 ± | 1.69 ± | 1.49 ± | 1.61 ± | 1.67 ± |
| (mm) | 0.06 | 0.03 | 0.09 | 0.07 | 0.05 | 0.07 | 0.07 | 0.05 |
| LVID; d | 3.15 ± | 3.07 ± | 3.85 ± | 3.04 ± | 3.05 ± | 3.21 ± | 3.81 ± | 3.39 ± |
| (mm) | 0.08 | 0.08 | 0.25 | 0.12 | 0.16 | 0.11 | 0.09 | 0.13 |
| LVID; s | 1.79 ± | 1.65 ± | 2.92 ± | 2.04 ± | 1.72 ± | 1.94 ± | 3.09 ± | 2.35 ± |
| (mm) | 0.09 | 0.07 | 0.14 | 0.07 | 0.15 | 0.08 | 0.12 | 0.13 |
| LVPW; d | 1.07 ± | 1.27 ± | 1.43 ± | 1.52 ± | 1.13 ± | 1.30 ± | 1.45 ± | 1.22 ± |
| (mm) | 0.04 | 0.08 | 0.07 | 0.06 | 0.06 | 0.11 | 0.08 | 0.09 |
| LVPW; s | 1.46 ± | 1.64 ± | 1.70 ± | 1.85 ± | 1.56 ± | 1.69 ± | 1.64 ± | 1.51 ± |
| (mm) | 0.05 | 0.07 | 0.06 | 0.07 | 0.08 | 0.11 | 0.08 | 0.10 |
| EF (%) | 75.4 ± | 78.9 ± | 47.2 ± | 62.5 ± | 76.4 ± | 71.3 ± | 39.6 ± | 59.2 ± |
| | 2.2 | 1.5 | 3.1 | 1.6 | 2.83 | 1.9 | 2.7 | 2.9 |
| FS (%) | 43.2 ± | 46.3 ± | 23.5 ± | 32.7 ± | 44.6 ± | 39.7 ± | 19.0 ± | 30.9 ± |
| | 1.9 | 1.5 | 1.9 | 1.1 | 2.6 | 1.6 | 1.5 | 1.9 |
| LV Mass | 125.7 ± | 141.9 ± | 219 ± | 183.9 ± | 133.5 ± | 146.9 ± | 238.6 ± | 168.9 ± |
| (mg) | 10.3 | 8.3 | 15.2 | 13.7 | 10.3 | 13.5 | 15.9 | 13.7 |
| LV Mass | 100.6 ± | 113.5 ± | 175.2 ± | 147.1 ± | 106.8 ± | 117.5 ± | 190.9 ± | 135.1 ± |
| (mg) | 8.3 | 6.6 | 12.2 | 11.0 | 8.2 | 10.8 | 12.7 | 10.9 |
| (corr.) | | | | | | | | |
| LV Vol; d | 39.8 ± | 37.3 ± | 67.2 ± | 36.7 ± | 37.9 ± | 42.0 ± | 62.6 ± | 47.8 ± |
| (μl) | 2.7 | 2.3 | 11.7 | 3.1 | 4.8 | 3.5 | 3.4 | 4.3 |
| LV Vol; s | 9.9 ± | 7.9 ± | 33.7 ± | 13.7 ± | 9.74 ± | 12.1 ± | 38.5 ± | 19.8 ± |
| (μl) | 1.4 | 0.7 | 4.1 | 1.2 | 2.00 | 1.3 | 3.5 | 2.6 |

The attenuation of pathological remodeling was further evidenced by decreased fibrosis in NR treated Sirt3Ko TAC mice (FIGS. 1H and 1I). Atrial natriuretic peptide (Nppa) and connective growth factor (Ctgf) levels were also decreased in NR treated Sirt3ko and WT TAC mice (FIG. 9D). Consistently, metabolites indicative of increased myocardial fibrosis (Sansbury et al., 2014), e.g. hydroxyproline, putrescine, and spermidine, were higher in myocardial tissue of vehicle treated TAC hearts, which was reduced by NR treatments (FIG. 8E). Markers of lipid oxidation (oxidized glutathione) were also decreased in TAC mice that received the NR treatments. (FIG. 8E). NR treatment improved cardiac dysfunction and held the progression of heart failure in both WT and Sirt3Ko mice.

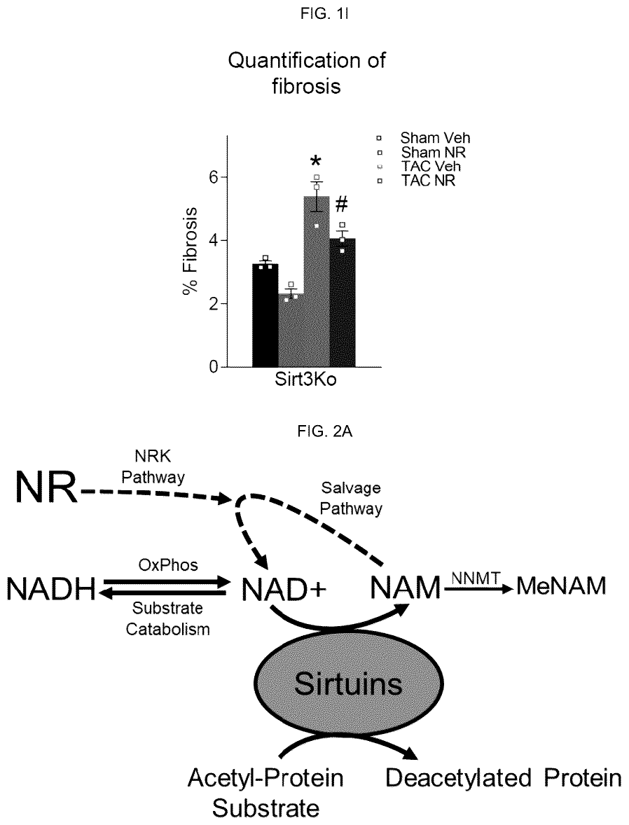

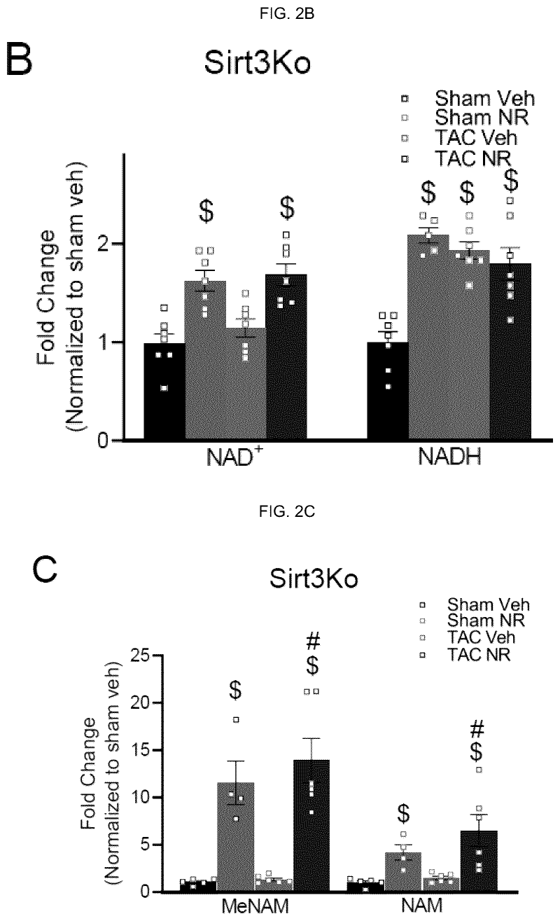

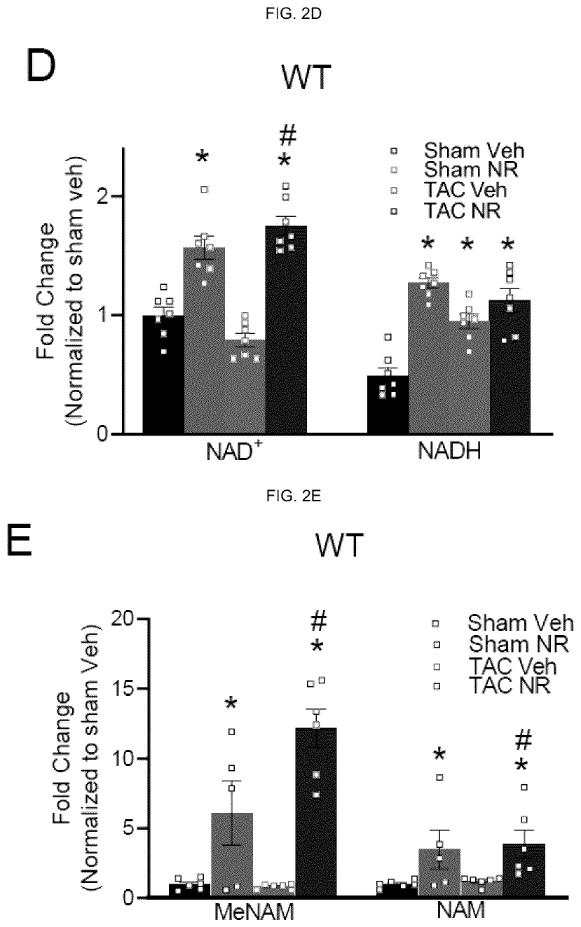

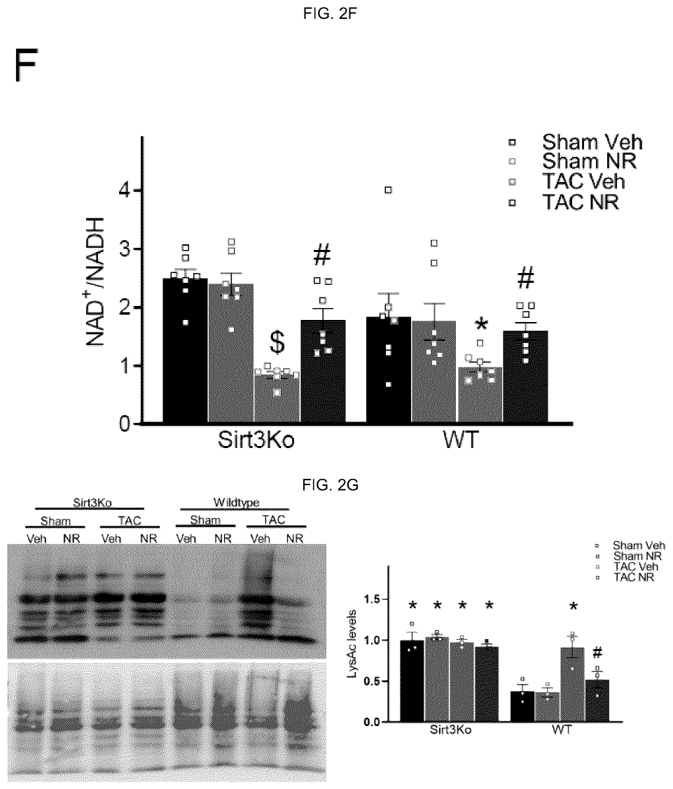

NR treatments increased cardiac levels of NAD(H), nicotinamide (NAM), and methyl-nicotinamide (MeNAM) in both WT and Sirt3Ko hearts (FIGS. 2A-2E), suggesting that NR stimulated the NAD⁺ salvage pathway in mouse hearts. NR administration normalized the NAD⁺/NADH ratio in both genotypes (FIG. 2F) and reversed the protein hyperacetylation in WT control mice after TAC (FIG. 2G). Despite the clear improvement in heart function and attenuation of multiple genetic and metabolic markers of heart failure, no change in mitochondrial protein acetylation was found in the NR treated Sirt3Ko mice even with restoration of redox imbalance (FIGS. 2F and 2G). Mitochondrial protein acetylation increased in Sirt3Ko compared to WT sham controls, but no further increases in lysine acetylation occurred when exposed to chronic pressure overload (FIG. 2G). In contrast, WT TAC hearts had robust increases in mitochondrial protein acetylation compared to sham controls (FIG. 2G). NR reduced TAC-induced increases in mitochondrial protein acetylation in WT but not Sirt3ko failing hearts (FIG. 2G). Taken together, the NR treatments improved contractile function, reduced LV dilation and cardiac hypertrophy in TAC-stressed mice whether Sirt3 was present or not (FIG. 1). The data suggest that efficacy of targeting NAD(H) level in heart failure could occur independent of Sirt3 mediated protein deacetylation. This mouse model, thus, provides an opportunity to determine cardioprotective mechanisms independent of mitochondrial protein deacetylation.

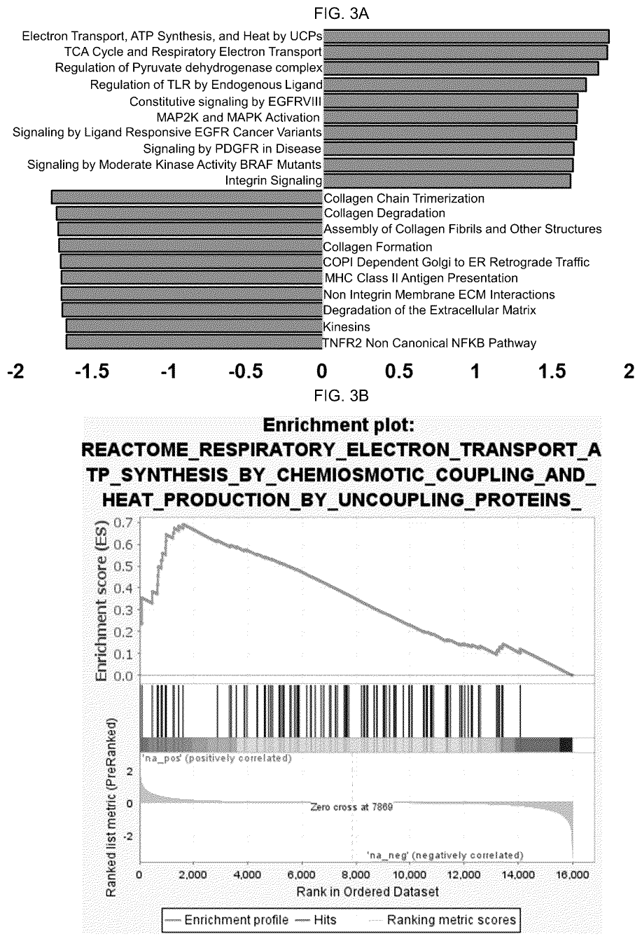

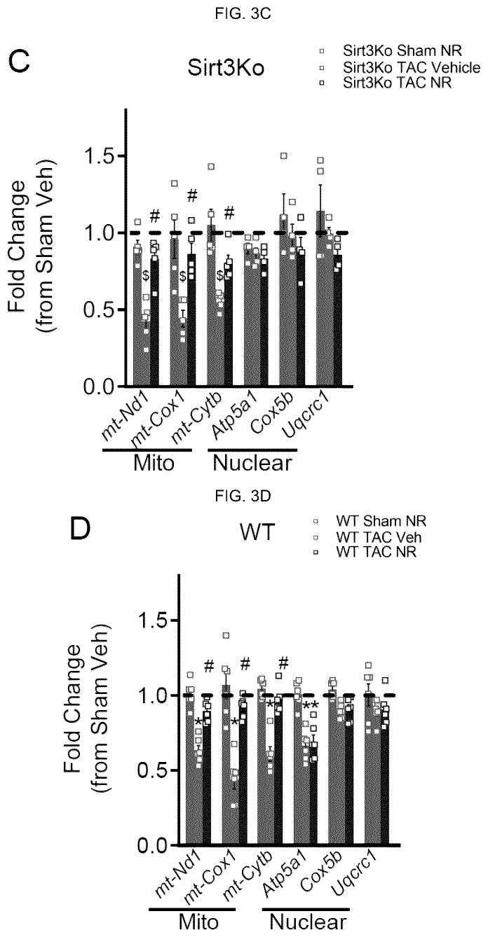

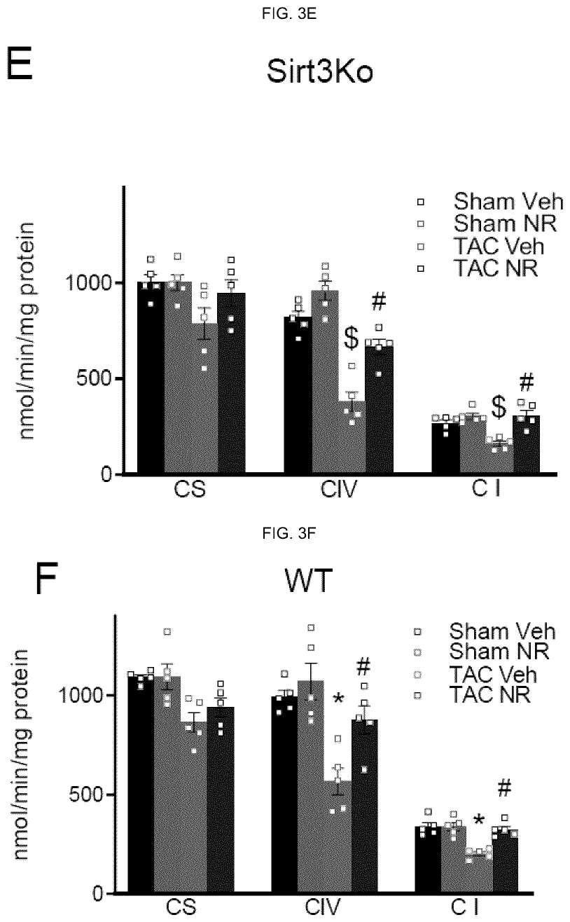

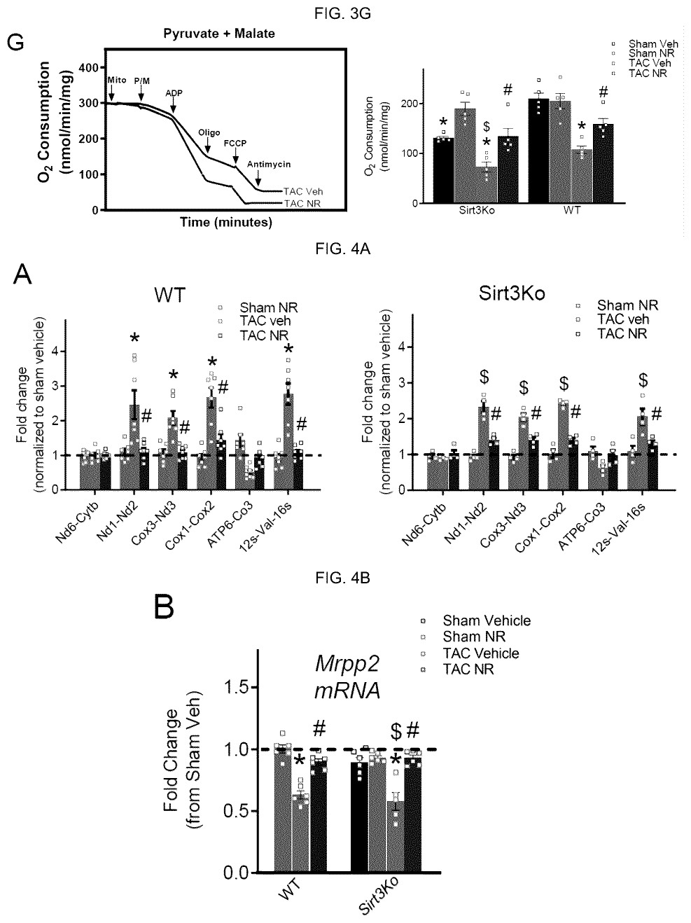

By RNA sequencing of TAC hearts treated with NR or vehicle, and examining the enrichment of genes that belong to known molecular pathways from the Reactome database using gene set enrichments analysis (GSEA), it is shown that genes significantly downregulated in NR treated TAC hearts were enriched in pathways relating to collagen formation and extracellular matrix organization while genes significantly upregulated in NR treated TAC hearts were enriched in pathways relating to mitochondrial metabolism (FIG. 3A). The top gene sets upregulated in NR treated TAC hearts included mitochondrial electron transport chain (ETC), mitochondrial biogenesis, Complex I biogenesis, BCAA catabolism, and fatty acid β-oxidation (FIG. 3A). The GSEA analysis demonstrated that the set of genes most heavily upregulated by NR treatment were mitochondrial DNA (mtDNA) encoded mRNA transcripts and uncoupling proteins (UCPs) (FIG. 3B), thereby demonstrating mechanisms that mediate the beneficial effects of administering an NAD precursor.

UCPs are known to be genetic markers of high rates of FAO in the heart and the expression level of UCPs are known to be regulated by PPARα in rodent hearts (Hilse et al., 2018; Young et al., 2001). Transcription of mtDNA directly affects mitochondrial respiration as all proteins encoded by mtDNA are subunits of ETC complexes (D'Souza and Minczuk, 2018; Karamanlidis et al., 2010). In both Sirt3Ko and WT groups, expressions of mtDNA encoded genes (mt-Nd1, mt-Cox1 and mt-Cytb) were significantly downregulated in TAC hearts, which was improved by NR treatment while nuclear DNA encoded ETC genes were not changed (FIGS. 3C and 3D). Similarly, the activities of cytochrome c oxidase (COIV) and complex I, of which both contain subunits encoded by mtDNA, decreased significantly in TAC hearts and were restored by NR treatment (FIGS. 3E and 3F). The activity of nuclear DNA encoded citrate synthase showed no change (FIGS. 3E and 3F). Mitochondrial respiratory function, assessed by State 3 respiration, was improved by NR treatment in both genotypes even though mitochondria remained hyperacetylation in Sirt3Ko hearts (FIG. 3G). These results suggest that mtDNA transcription could be a mechanism responsible for the beneficial effects of NR treatment.

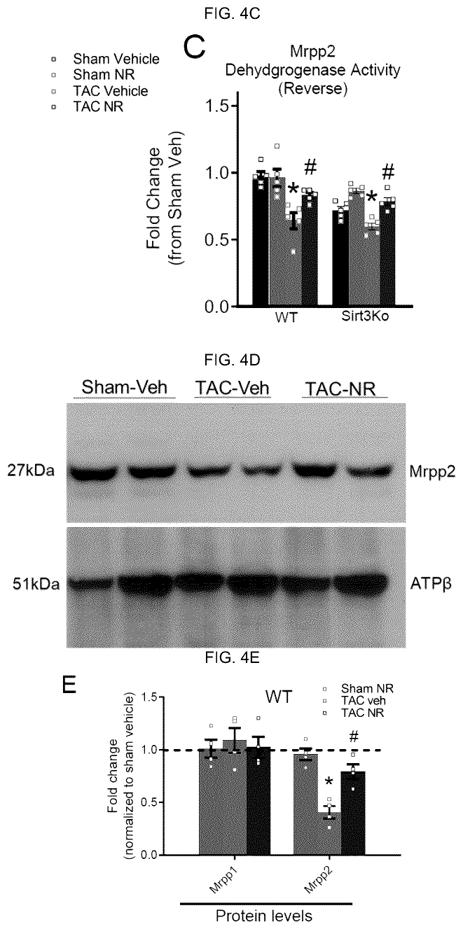

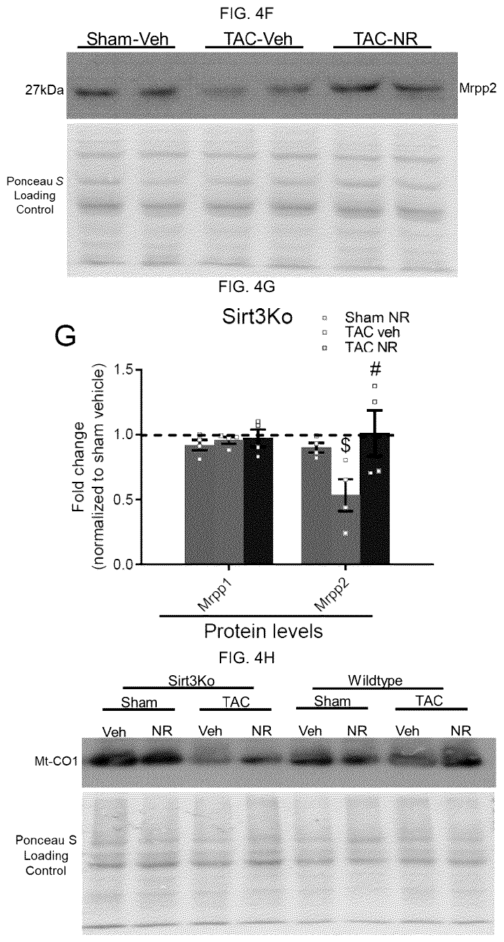

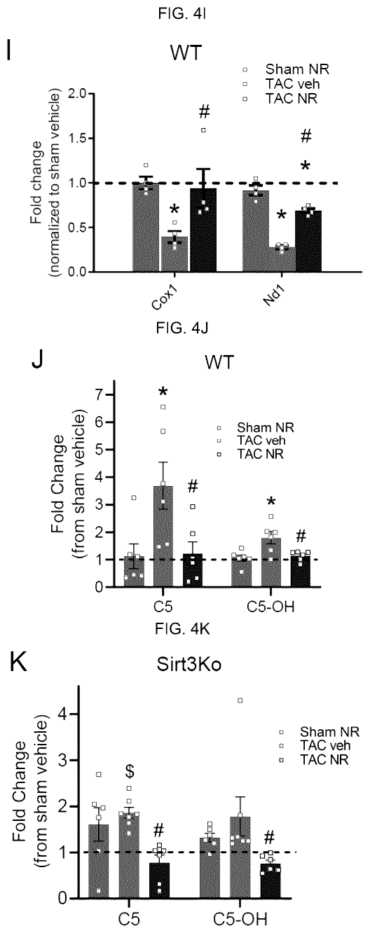

Transcription of mitochondrial DNA (mtDNA) in failing mouse hearts +/−NR treatments was determined. Mitochondrial genome is transcribed as long polycistronic transcripts in which large majority of mRNA and rRNA elements are flanked by one or more tRNA elements. Excision of the tRNA elements by mitochondrial ribonuclease P (RNase P) and RNase Z endonucleases are required for RNA maturation (Bhatta et al., 2021; D'Souza and Minczuk, 2018; Holzmann et al., 2008; Kotrys and Szczesny, 2020; Rackham et al., 2016). It was found that the pre-RNA fragments, e.g. Nd1-Nd2 or Cox1-Cox2, increased 2-3 folds in failing hearts of both WT and Sirt3Ko mice (FIG. 4A). Such change in the transcript pattern indicated reduced function of RNase P complex (Rackham et al., 2016). NR treatment removed the accumulation of RNA precursor fragments suggesting that the improvement involved a NAD(H)-dependent mechanism (FIG. 4A).

Among the RNase P subunits, Mrpp2 is a multifunctional protein that possess NAD(P)(H)-dependent short chain dehydrogenase/reductase (SDR) activity in addition to forming a complex with Mrpp1 for RNase P reaction.

Expression levels of Trmt10c and Hsd17b10, which encode protein 1 (Mrpp1) and protein 2 (Mrpp2) of the RNase P complex, respectively, were determined. Trmt10c levels were not different among any comparisons while the Hsd17b10 transcripts were decreased in TAC hearts (FIG. 9A and FIG. 4B).

Severe accumulations of C5:1(tiglyl-)carnitine and C5-OH-(2-methyl-3-hydroxybutyryl-)carnitines, which are known to increase in patients suffering from Hsd17b10 deficiency (Akagawa et al., 2017; Falk et al., 2016; Fukao et al., 2014; Oerum et al., 2017; Sutton et al., 2003; Zschocke, 2012; Zschocke et al., 2000), were determined (FIGS. 4J and 4K). These changes suggested an impairment of NAD(H) coupled shortchain dehydrogenase/reductases function of Mrpp2 in TAC hearts. In support of this, Mrpp2 dehydrogenase activity was significantly decreased in TAC heart tissues of both WT and Sirt3Ko mice (FIG. 4C). Western blot analysis confirmed the deficiency of Mrpp2 in failing hearts with no change in Mrpp1 levels, suggesting that Mrpp2 deficiency is the culprit in disrupting RNase P activity in failing hearts (FIGS. 4D-4G).

When NR was provided to the failing hearts as an NAD precursor, normalization of NAD⁺/NADH ratio was accompanied by ˜2-fold increase in Mrpp2 dehydrogenase activity matching the function of the sham operated controls (FIG. 4C). The normalization of Mrpp2 protein level (FIG. 4C-4G) and dehydrogenase activity in the NR treated TAC hearts suggested that restoration of NAD(H) redox homeostasis in failing hearts could improve both the catalytic and non-catalytic functions of Mrpp2. In order to confirm that the mtDNA transcript level changes were responsible for OXPHOS protein expression, protein level of complex I and IV subunits was analyzed, observing marked reduction in mt-Cox1 and in mt-Nd1 protein expression in TAC hearts (FIGS. 4H-41 and FIG. 9A). The protein levels of mt-Cox1 and mt-Nd1 were preserved in TAC hearts that received NR treatments (FIGS. 4H-41 and FIG. 9B). These results demonstrated a novel mechanism for impaired mitochondrial biogenesis in the failing heart which extended beyond the widely recognized downregulation of PGC1α mediated transcription.

In addition to the ability of NR to increase intracellular NAD⁺ levels in failing hearts and preserve Mrpp2 functions, the role of other members of the NAD(P)(H)-dependent short-chain dehydrogenase/reductases (SDR) family in heart failure was determined. The SDR family of enzymes are NAD(P)(H)-dependent oxidoreductases involved in the metabolism of a large variety of compounds, including steroid hormones, prostaglandins, retinoids, lipids, and xenobiotics (Belyaeva et al., 2008; Kallberg et al., 2010; Kavanagh et al., 2008; Pares et al., 2008). Many members of the SDR family of proteins are strongly expressed in the heart and display distinct preferences and binding affinities for either NAD(H) or NADP(H) (Kavanagh et al., 2008; Roussel et al., 2018). Most SDR's functioning in oxidative pathway's prefer NAD(H) as a cofactor while SDR's functioning in reductive pathway's prefer NADP(H) (Kallberg et al., 2010; Kavanagh et al., 2008).

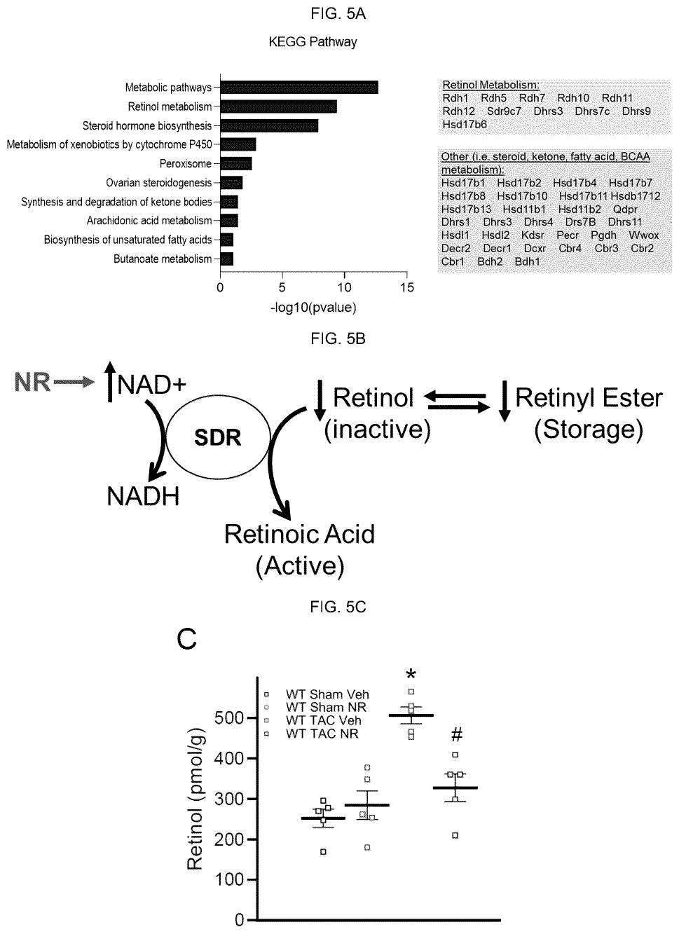

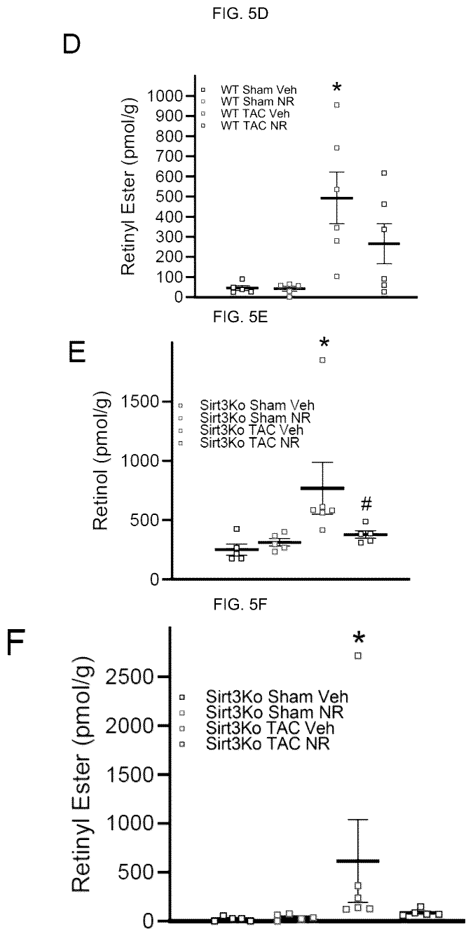

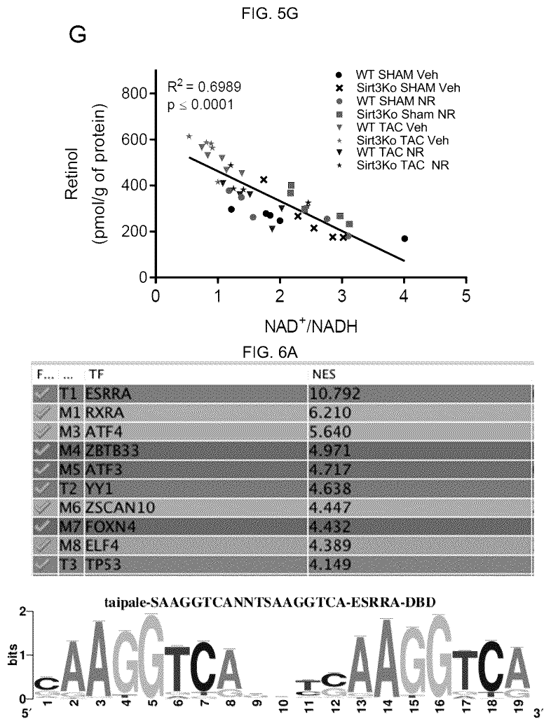

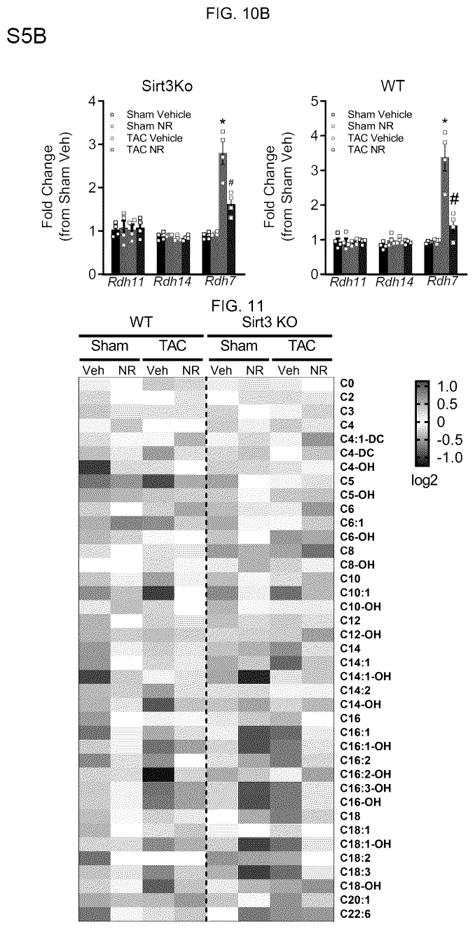

NAD(P)(H)-dependent SDR's have inhibited functions in failing hearts due to chronic redox imbalance (NAD⁺/NADH). RNAseq data was annotated to classify genes into protein families (Supplemental File S1). The list was annotated using the Universal Protein Resource (Uniprot). Search parameters returned 43 genes from sham and TAC mouse heart belonging to the SDR family including the Hsd17b10 gene (FIG. 5A, right green and blue box). The list was further annotated by Gene Ontology in the Database for Annotation, Visualization and Integrated Discovery (DAVID) (FIG. 10A). Eighteen gene ontology categories were identified in the analysis. Oxidoreductase activity and retinol dehydrogenase activity were the most significantly enriched molecular functions (FIG. S5A). Pathway analysis (KEGG Pathway) revealed retinol metabolism as the most enriched category stemming from the SDR gene set (FIG. 5A, left). The search identified eleven SDRs involved in retinol metabolism (FIG. 5A, right green box).

In particular, NAD(P)(H)-dependent SDR members that control rate-limiting steps in retinoid biosynthesis and that are highly expressed in mouse heart are impacted by redox imbalance. Retinol and its storage form (retinyl-palmitate) in TAC hearts from Sirt3Ko and WT hearts +/−NR were quantified, demonstrating robust accumulations of retinol and retinylpalmitate in the failing hearts of both genotypes (FIG. 5B-5F). Increased retinol was due to decreased activity of the redox sensitive SDRs involved in retinol oxidation thus created a “bottleneck” (FIG. 5B). Tissue retinol concentrations significantly correlated with the observed intracellular NAD⁺/NADH ratio (FIG. 5G). Reductions of NAD⁺/NADH in TAC hearts resulted in higher concentrations of retinol (FIG. 5G). Administering NAD precursors in the TAC hearts removed the “bottleneck” in the SDRs reactions in both WT and Sirt3Ko mice (FIGS. 5C-5F). In addition, decreased expression of the dehydrogenases was not responsible for the stalled reaction. Transcript levels of retinol dehydrogenase 11 (Rdh11) and Rdh14 were not different in failing hearts (FIG. 10B). In contrast, there was a robust induction of Rdh7 (Crad2) in both WT and Sirt3Ko failing hearts (FIG. 10B). These results suggested that NAD(H) redox sensitive SDRs, especially retinol oxidation enzymes, are likely targets of NR treatment.

The products of retinol oxidation, retinoic acid (RA) and its isomers, are considered key endogenous ligands of retinoic acid receptor/retinoid X receptor (RAR/RXR). In the heart, RXRα forms complex with PPARα to regulate the transcription of proteins for oxidative metabolism, especially fatty acid oxidation (Kelly, 2003; Yang et al., 2021). The PPARα/RXRα transcriptional cascades are known to be downregulated and thought to be maladaptive in failing heart (Finck, 2007; Kelly, 2003).

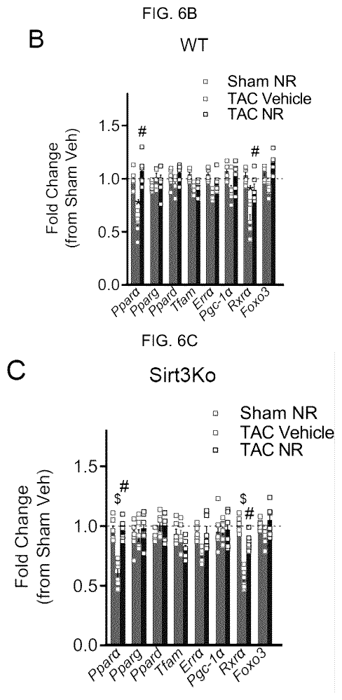

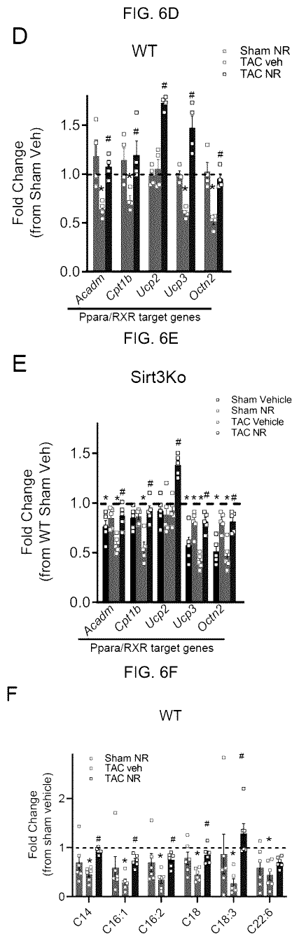

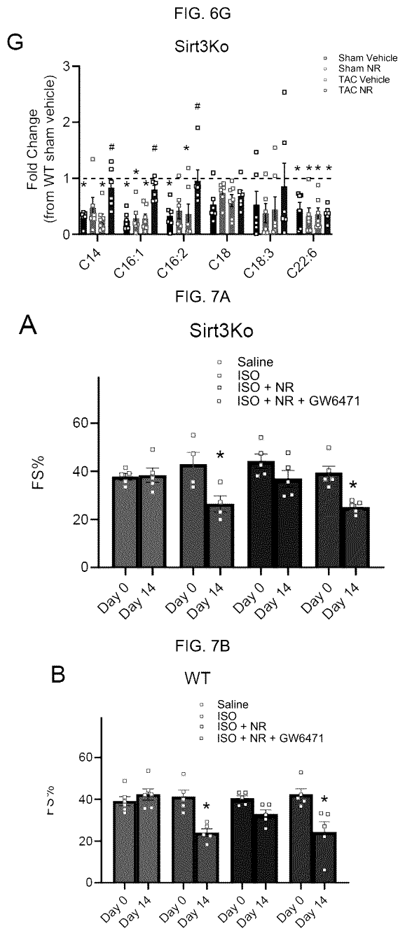

Thus, restoring retinol oxidation, the main pathway for RA biosynthesis, would increase RXRα ligand availability and improve the transcriptional activity of the PPARα/RXRα complex. RNAseq dataset was analyzed to determine transcription factor (TF) motifs enriched in genes upregulated in NR treated TAC hearts. Genes from upregulated pathways in the GSEA analysis (defined by a family-wise error rate [FWER]<0.25) were further analyzed using iRegulon (Janky et al., 2014). TF's/Motifs identified are clustered by similarity and ranked by normalized enrichment score (NES) (FIG. 6A). RXRα was one of the top 3 TF's identified among the upregulated genes in NR vs vehicle treated TAC hearts (FIG. 6A, top). The highest NES scores were motifs containing the consensus sequence AGGTCA which are known binding sites for RXRα and its interaction partner PPARα (FIG. 6A, bottom).

Furthermore, PPARα/RXRα signaling was more active in NR treated TAC hearts. Parallel RT-PCR analysis showed that Rxrα and its interaction partner Pparα expression were profoundly decreased in the failing hearts regardless of genotype (FIGS. 6B and 6C). The expression of other transcriptional regulators including Pgc1-α or Tfam showed very modest change (FIGS. 6B and 6C). As previously reported (Sack and Kelly, 1998; Sack et al., 1996), expression levels PPARα/RXRα target genes were decreased in TAC hearts (FIGS. 6D and 6E). The selected target genes in our analysis represented a wide range of lipid metabolism genes for the transport, storage, and oxidation of fatty acids.

Both the organic cation/carnitine transporter 2 (Octn2) and the Carnitine Opalmitoyltransferase I, muscle isoform (Cpt1b) are regulated by PPARα/RXRα (Barger et al., 2000; Bedi et al., 2016; Luo et al., 2014; Maeda et al., 2008; van Vlies et al., 2007; Wen et al., 2010). Octn2 is required for cellular carnitine uptake and Cpt1b for mitochondrial uptake of long-chain fatty acids, respectively (Barger et al., 2000; Bedi et al., 2016). Decreased expression of the high affinity carnitine transporter (Octn2) and the rate-limiting enzyme (Cpt1b) in mitochondrial (p)-oxidation would lower myocardial acylcarnitine's in failing hearts. Indeed, acylcarnitine (AC) profiling of the failing hearts showed that most lipid derived acylcarnitines were decreased, whereas amino acid markers were increased in both genotypes (FIGS. 6F and 6G and 11A). This profile was consistent with changes observed in human failing hearts (Bedi et al., 2016; Murashige et al., 2020; Previs et al., 2022) and implied a metabolic switch away from (β)-oxidation in mitochondria and diminished degradation of amino acids.

WT TAC hearts have low levels of free carnitine (CO) highly suggestive of Octn deficiency (FIG. 11A). Both genotypes had decreased palmitoylcarnitine (C16) and stearoylcarnitine (C18) in TAC (FIGS. 6F and 6G), which are known proxies of their respective acyl-CoA derivates that formed in mitochondrial β-oxidation. NR treatment increased the expression of PPARα target genes, e.g. medium-chain specific acyl-CoA dehydrogenase (Acadm), Ucp2, Ucp3, Octn2, and Cpt1b in the failing hearts. Tissue content of lipid derived acylcarnitines and hydroxy-acylcarnitines increased in NR treated TAC hearts to the level similar to that of WT-Sham hearts (FIGS. 6F-G and 11A). C16 levels increased in WT and Sirt3ko NR treated TAC hearts compared to vehicle (FIGS. 6F-G). Overall, NR treated TAC hearts presented a genetic and metabolic profile more closely resembling that of fatty acid oxidation in sham-operated hearts. These findings suggested that PPARα/RXRα activation was likely at the center of the NR effects regardless of mitochondrial protein acetylation status.

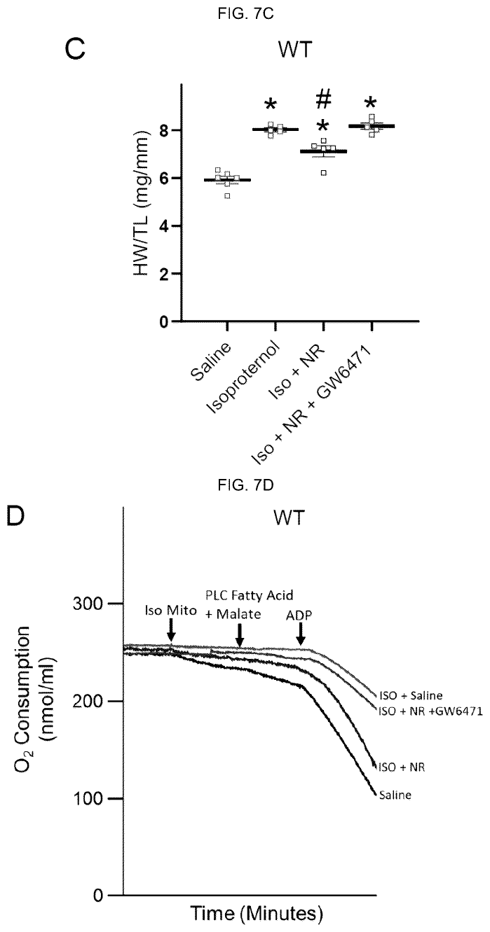

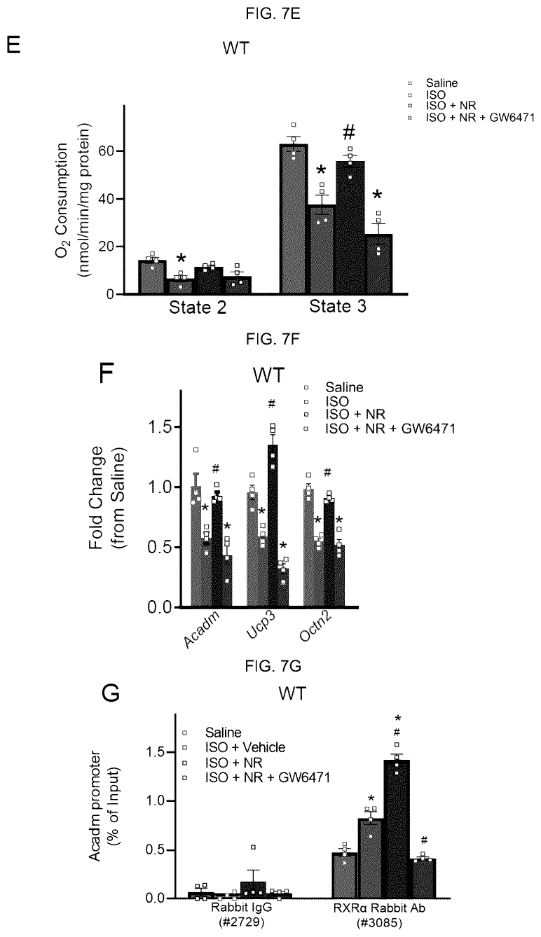

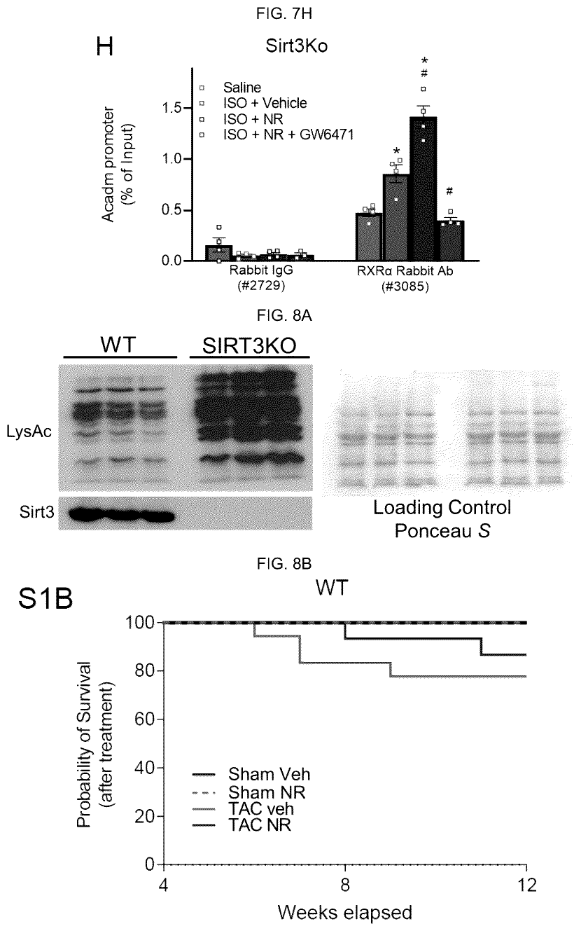

The inhibition of PPARα/RXRα signaling complex by the selective inhibitor GW6471 blocks the therapeutic effects of NR in mice subjected to chronic isoproterenol (ISO) stress. Chronic ISO caused significant cardiac hypertrophy and declines in FS % as assessed by murine echocardiography (FIGS. 7A-C and 12A-C).

Consistent with the results obtained from TAC hearts in FIGS. 1A-1J, NR treatment increased FS % and partially reduced cardiac hypertrophy in mice subjected to 14 days of ISO. This protection was blocked by treatment with GW6471. The role of mitochondrial oxidative metabolism was evaluated by measuring mitochondrial oxygen consumption using the fatty acid substrate palmitoylcarntine (PLC). PLC supported State 3 respiration (ADPstimulated) was decreased in cardiac mitochondria isolated from ISO treated hearts of both genotypes, which was restored by NR treatments (FIGS. 7D-7E and FIG. 12B). This response was clearly blocked when mice received GW6471 in addition to NR (FIGS. 7D-7E and FIGS. 12A-C). Furthermore, NR in the presence of GW6471 failed to preserve PPARα/RXRα target gene expression in ISO treated hearts (FIGS. 7F and 12C). Utilizing chromatin immunoprecipitation (ChIP) it was found that NR treatment increased binding occupancy of RXRα to the PPARα response element (PPRE) in the Acadm promoter in ISO mouse hearts. This response could be blocked by the selective inhibitor GW6471 (FIGS. 7G and 7H). Taken together, these observations strongly supported the notion that NR treatment activated RXRα in the hearts, and furthermore, the activation of PPARα/RXRα signaling cascade was required for the benefit observed.

Accordingly, before the current disclosure, it was not known and could not have been reasonably predicted that administering NAD precursor blunts the development of heart failure independent of mitochondrial protein deacetylation in mice subjected to two forms of pathological stresses. These observations open doors for new mechanisms of action in NAD⁺ targeted therapy.

NADH redox sensitive SDR family proteins are identified as important regulators of mitochondrial biogenesis and fatty acid oxidation. Previously, downregulation of PGC1α mediated mitochondrial biogenesis has been considered a main mechanism for impaired OXPHOS in failing hearts (Lehman et al., 2000). However, PGC-1α downregulation does not account for decreases in the expression of mtDNA encoded proteins in human failing heart, and moreover, promoting PGC-1α driven mitochondrial biogenesis in animal models of HF does not rescue the mitochondrial dysfunction or improve the outcome (Karamanlidis et al., 2014; Karamanlidis et al., 2010; Lehman et al., 2000).

A significant defect in mtDNA post-transcriptional processing by mitochondrial RNase P complex suggests a new mechanism to account for low mtDNA-encoded transcripts in the failing hearts. The protein responsible for the defective function of mitochondrial RNase P complex, Mrpp2, also functions as a NAD(P)(H) dependent SDR in BCAA catabolism (Holzmann et al., 2008; Sun et al., 2016; Zschocke, 2012). Suppression of BCAA catabolic genes with concomitant increase in tissue BCAAs has been previously reported (Sun et al., 2016). Patients with Hsd17b10 mutations, which encodes Mrpp2, suffer from mitochondrial disease with low expression of mtDNA-encoded proteins, elevated levels of BCAAs, and early fatality (Falk et al., 2016; Zschocke et al., 2000).

Thus, Mrpp2 is a determinant and a therapeutic target for mitochondrial dysfunction. While unbiased transcriptome analysis in our study shows that Hsd17b10 deficiency in the failing heart can be corrected with NR administration, future studies are required to determine how administering NAD precursor restores the Mrpp2 protein level.

A hallmark of metabolic remodeling in heart failure is decreased fatty acids oxidation, largely attributed to the downregulation of PPARα gene expression and activity (Finck, 2007; Lopaschuk et al., 2021; Lopaschuk et al., 2010; Zhou and Tian, 2018). However, targeting PPARα by pharmacological activators or cardiac-specific overexpression has not translated into therapy (Finck et al., 2002; Karamanlidis et al., 2014; Lehman et al., 2000; Sambandam et al., 2006). PPARα regulates the expression of key components of fatty acid uptake, esterification, and oxidation through the nuclear transcriptional complex with RXRα (Finck, 2007; Kelly, 2003). The present bioinformatics analysis points to activation of RXRα by NR treatment. While endogenous ligands for RXRα activation are difficult to detect in adult murine heart, it is generally accepted that isomers of retinoic acid, which are generated downstream of retinol oxidation, are primary ligands.

Retinol oxidation is stalled and activation of SDRs in retinol metabolism pathway restores PPARα/RXRα signaling in the failing mouse hearts. In support, accumulation of retinol in failing hearts has been reported in humans and in guinea pigs although the exact mechanisms are not clear (Yang et al., 2021). Thus, imbalanced intracellular redox negatively impacts NAD(P)(H)-dependent SDRs involved in retinol oxidation, and that normalization of NAD⁺/NADH ratio by providing NR as an NAD precursor can remove the “bottleneck” in retinol oxidation in failing heart and increase local ligand availability for RXRα.

The retinoid acid (RA) isomers that activate RXRα in vivo could not be detected. Direct measurement of natural ligands of RXRα in vivo remains a challenge in the field, particularly for adult hearts (de Lera Á et al., 2016; Niu et al., 2017; Yang et al., 2021). New analytical tools will be required to determine the exact isomer of RA that increases with NR to activate RXRα in the heart.

In summary, administering NAD precursor in failing hearts improves mitochondrial function via both mitochondrial and nuclear transcriptional processes. These mechanisms allow for a coordinated increase in OXPHOS and FAO independent of Sirt3 mediated protein deacetylation. Furthermore, prior results of NAD⁺ boosting in preventing cardiac dysfunction are extended by demonstrating the efficacy of treatment in mice with established pathologic hypertrophy and myocardial dysfunction. By a previously unknown mechanism by which mtDNA encoded proteins is decreased in the failing heart, strategies aiming at stabilizing RNase P complex formation and activity could be a new therapeutic target to alleviate mitochondrial dysfunction. Finally, targeting SDR proteins in heart failure could impact many important pathways known to be dysregulated in failing heart including steroid, prostaglandins, retinoids, lipids, and mtDNA post-transcriptional processing.

Aspects of the disclosure are now described in additional detail as follows: (i) Human Subjects with Cardiac Dysfunction, (ii) NAD Precursors, (iii) Compositions for Administration, (iv) Methods of Use, (v) Experimental Example 1, (vi) Experimental Example 2, and (vii) Closing Paragraphs.

(i) Human Subjects with Cardiac Dysfunction. Part of the current disclosure includes identifying subjects with cardiac dysfunction. Cardiac dysfunction as used herein refers to a chronic, progressive condition in which the heart muscle is unable to pump enough blood to meet the subject's needs for blood and oxygen. Cardiac dysfunction is a clinical disorder characterized by congestion and decreased functional capacity. Cardiac dysfunction may include the left side, right side, or both sides of the heart. Accordingly, in some embodiments, cardiac dysfunction may be left-sided heart failure (systolic failure or diastolic failure), right-sided heart failure, congestive heart failure, or any combination thereof.

Heart failure may be classified by the New York Heart Association (NYHA) Functional Classification system. The NYHA Functional Classification system may include a functional capacity, which is a description of how the subject feels during physical activity, and an objective assessment. Functional capacity is ranked from class I to class IV, with increasing limitations of physical activity. Objective assessment is ranked from class A to class D, with increasing severity. Four stages of cardiac dysfunction or heart failure have been identified to classify the evolution and progression of the disease. Stage A and Stage B are considered pre-heart failure where Stage A refers to a patient at high risk for developing heart failure but does not have a structural disorder of the heart. Stage A patients may have a family history of heart failure or may have a medical condition including hypertension, diabetes, coronary artery disease, metabolic syndrome, or may have a history of alcohol abuse, rheumatic fever, or drug use. Stage B refers to a patient with a structural disorder of the heart but has not developed symptoms of heart failure. For example, a Stage B patient could be diagnosed with systolic left ventricular dysfunction but does not have any symptoms of heart failure. Traditional clinical diagnosis for heart failure includes Stage C and Stage D. Stage C refers to a patient with underlying structural heart disease that has past or current symptoms of heart failure the condition. In certain examples, Stage D refers to a patient with final stage of the disease who requires specialized treatment strategies such as mechanical circulatory support, continuous inotropic infusions, cardiac transplantation, heart surgery, or palliative or hospice care.

(ii) NAD Precursors. As used herein, NAD precursors increase levels of NAD in myocardial tissue following administration of an effective dose.

In particular embodiments, the NAD precursor is NR (CAS Number 1341-23-7). The chemical structure of NR includes

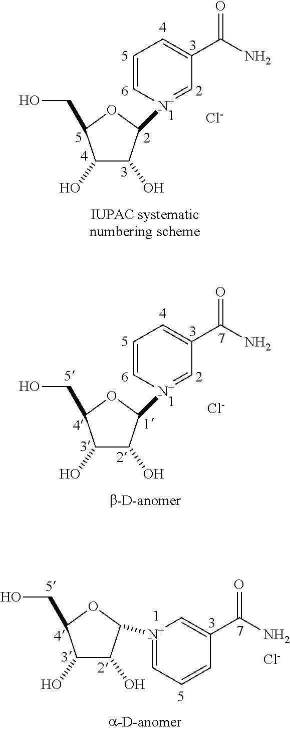

NR can be isolated from natural sources or synthesized. Exemplary isolation and synthesis methods are described in Haynes et al., A. J. Chem. Soc. 1957, 3727-3732 and WO 2010/111111. In particular embodiments, NR can be used in its reduced form (NRH) as a 1,4-dihydropyridine compound. Nicotinic acid riboside (NAR) and its reduced form (NARH) can also be used as NAD precursors. For example, US20180362570 describes oxidized and reduced forms of NR with improved stability and bioavailability compared to NR. Particularly described are compounds MP-05, MP-06, MP-07, MP-08, MP-09 and MP-10.

US20180200275 describes that mixing NR with anthocyanin(s) or flavan-3-ol(s) of flavonoids forms positively-charged aggregating molecular forms through co-solvation and improves the oral absorption of NR through the stomach or intestine.

Useful NR derivatives are described in U.S. Ser. No. 10/000,520 and US20060229265.

U.S. Ser. No. 10/000,520 describes a structure:

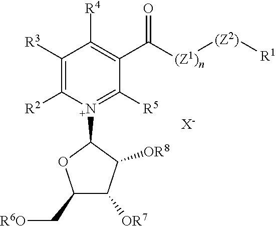

wherein X⁻ is selected from the group including fluoride, chloride, bromide, iodide, formate, acetate, ascorbate, benzoate, carbonate, citrate, carbamate, formate, gluconate, lactate, methyl bromide, methyl sulfate, nitrate, phosphate, diphosphate, succinate, sulfate, trifluoromethanesulfonate, trichloromethanesulfonate, tribromomethanesulfonate, and trifluoroacetate;
Z¹ and Z² are independently NH or oxygen;
n is 0 or 1;
R¹ is selected from the group including hydrogen, substituted or unsubstituted (C₁-C₃)alkyl, substituted or unsubstituted (C₁-C₃)cycloalkyl, substituted or unsubstituted aryl, substituted or unsubstituted heteroaryl, and substituted or unsubstituted heterocycle, vitamin 1 ester, vitamin B2 ester, vitamin B6 ester, and —C**H—(R^{A})—CO₂R^{B}; wherein the substituted (C₁-C₃)alkyl, substituted (C₁-C₃)cycloalkyl, substituted aryl, substituted heteroaryl, and substituted heterocycle are substituted with one to five substituents independently selected from the group including —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
R^{A} is selected from the group including —H, —(C₁-C₆)alkyl, —(CH₂)₃—NH—C(NH₂)(═NH), —CH₂C(═O)NH₂, —CH₂COOH, —CH₂SH, —(CH₂)₂C(═O)—NH₂, —(CH₂)₂COOH, —CH₂-(2-imidazolyl), —CH(CH₃)—CH₂—CH₃, —CH₂CH(CH₃)₂, —(CH₂)₄—NH₂, —(CH₂)₂—S—CH₃, phenyl, —CH₂-phenyl, —CH₂—OH, —CH(OH)—CH₃, —CH₂-(3-indolyl), —CH₂-(4-hydroxyphenyl), —CH(CH₃)₂, and —CH₂—CH₃;
R^{B} is hydrogen or —(C₁-C₃)alkyl;
each R^{C} is independently selected from the group including hydrogen, —(C₁-C₃)alkyl, substituted or unsubstituted pyridyl, substituted or unsubstituted 1,4-dihydropyridyl, a radical of the compound of formula (1), and vitamin B7 ester (biotinyl); wherein the substituted pyridyl and substituted 1,4-dihydropyridyl are substituted with one to five substituents independently selected from the group including —(C₁-C₆)alkyl, —(C₂-C₆)alkenyl, —(C₂-C₆)alkynyl, halogen, —CN, —NO₂, —C(O)R^{B}, —C(O)OR^{B}, —C(O)NR^{B}₂, —C(═NR^{B})NR^{B}₂, —OR^{B}, —OC(O)(C₁-C₆)alkyl, —OC(O)O(C₁-C₆)alkyl, —OC(O)NR^{B}₂, —(C₁-C₆)alkylene-NR^{B}₂, —NR^{B}₂, —NR^{B}C(O)R^{B}, —NR^{B}C(O)O(C₁-C₆)alkyl, —NR^{B}C(O)NR^{B}₂, —NR^{B}SO₂NR^{B}, —SR^{B}, —S(O)R^{B}, —SO₂R^{B}, —OSO₂(C₁-C₆)alkyl, —SO₂NR^{B}₂, —(C₁-C₆)perfluoroalkyl, and —(C₁-C₆)alkylene-OR^{B};
R², R³, R⁴, and R⁵ are each independently selected from the group including hydrogen, —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
R⁶ is selected from the group including hydrogen, —C(O)R′, —C(O)OR′, —C(O)NHR′, —P(O)(OY¹)(OY²), —P(O)(OY¹)(NHR″), substituted or unsubstituted (C₁-C₃)alkyl, substituted or unsubstituted (C₁-C₃)cycloalkyl, substituted or unsubstituted aryl, substituted or unsubstituted heteroaryl, and substituted or unsubstituted heterocycle, vitamin 1 ester, vitamin B2 ester, vitamin B6 ester, and —C**H—(R^{A})—CO₂R^{B}; wherein the substituted (C₁-C₈)alkyl, substituted (C₁-C₃)cycloalkyl, substituted aryl, substituted heteroaryl, and substituted heterocycle are substituted with one to five substituents independently selected from the group including —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, (C₁-C₆)alkylene-NR^{C}₂, —N₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
R′ is selected from the group including hydrogen, substituted or unsubstituted —(C₁-C₃)alkyl, substituted or unsubstituted —(C₁-C₃)cycloalkyl, substituted or unsubstituted aryl, substituted or unsubstituted heteroaryl, substituted or unsubstituted heterocycle, vitamin 1 ester, vitamin B2 ester, vitamin B6 ester, choline ester, biotin ester, vitamin A ester, resveratrol ester, aryl(C₁-C₄)alkyl, heterocycle(C₁-C₄)alkyl, and —C**H—(R^{A})—CO₂R; wherein the substituted (C₁-C₃)alkyl, substituted (C₁-C₃)cycloalkyl, substituted aryl, substituted heteroaryl, and substituted heterocycle are substituted with one to five substituents independently selected from the group including —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
R″ is selected from the group including hydrogen, substituted or unsubstituted (C₁-C₈)alkyl, substituted or unsubstituted (C₁-C₃)cycloalkyl, substituted or unsubstituted aryl, substituted or unsubstituted heteroaryl, and substituted or unsubstituted heterocycle, vitamin 1 ester, vitamin B2 ester, vitamin B6 ester, choline ester, biotin ester, vitamin A ester, resveratrol ester, aryl(C₁-C₄)alkyl, heterocycle(C₁-C₄)alkyl, and —C**H—(R^{A})—CO₂R; wherein the substituted (C₁-C₈)alkyl, substituted (C₁-C₃)cycloalkyl, substituted aryl, substituted heteroaryl, and substituted heterocycle are substituted with one to five substituents independently selected from the group including —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, (C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
R⁷ and R³ are independently selected from the group including hydrogen, —C(O)R′, —C(O)OR′, —C(O)NHR′, substituted or unsubstituted (C₁-C₃)alkyl, substituted or unsubstituted (C₁-C₃)cycloalkyl, substituted or unsubstituted aryl, substituted or unsubstituted heteroaryl, substituted or unsubstituted heterocycle, substituted or unsubstituted aryl(C₁-C₄)alkyl, and substituted or unsubstituted heterocycle(C₁-C₄)alkyl; wherein the substituted (C₁-C₃)alkyl, substituted (C₁-C₃)cycloalkyl, substituted aryl, substituted heteroaryl, substituted heterocycle, substituted aryl(C₁-C₄)alkyl, and substituted heterocycle(C₁-C₄)alkyl are substituted with one to five substituents independently selected from the group including —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
Y¹ and Y² are independently selected from the group including hydrogen, sodium, potassium, lithium, substituted or unsubstituted (C₁-C₃)alkyl, substituted or unsubstituted (C₁-C₈)cycloalkyl, substituted or unsubstituted aryl, substituted or unsubstituted heteroaryl, and substituted or unsubstituted heterocycle; wherein the substituted (C₁-C₃)alkyl, substituted (C₁-C₈)cycloalkyl, substituted aryl, substituted heteroaryl, and substituted heterocycle are substituted with one to five substituents independently selected from the group including —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, (C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C}; or, alternatively, Y¹ and Y² taken together are selected from the group including sodium, potassium, lithium, magnesium, calcium, strontium, and barium;
provided that when Z² is NH, the absolute configuration of C** is R or S, or a mixture of R and S;
further provided that when n is 0, Z² is NH, and R¹ is hydrogen, then R⁶, R⁷, and R⁸ are not all simultaneously hydrogen;
further provided that when n is 0, Z² is oxygen, and R¹ is hydrogen, then R⁶, R⁷, and R⁸ are not all simultaneously hydrogen, acetyl, or benzoyl; and further provided that when n is 0, Z² is oxygen, and R¹ is —(C₁-C₃)alkyl, then R⁶, R⁷, and R³ are not all simultaneously acetyl or benzoyl.

One example of an NR analogue includes the NR chloride (3-carbamoyl-1-[(2R,3R,4S5R)-3,4-dihydroxy-5-(hydroxymethyl)oxolan-2-yl]-pyrin-1-ylium chloride; also referred to as 1-(β-D-Ribofuranosyl) nicotinamide chloride, which is a salt form of NR; see US20170210774).

US20170204131 describes crystalline forms of NR chloride that are chemically stable. These crystalline forms of NR chloride include 3-carbamoyl-1-((2R,3R,4S,5R)-3,4-dihydroxy-5(hydroxymethy)tetrahydrofuran-2-yl)pyridin-1-ium(β-D-NR) chloride crystal, 3-carbamoyl-1-((2R,3R,4S,5R)-3,4-dihydroxy-5(hydroxymethy)tetrahydrofuran-2-yl)pyridin-1-ium(β-D-NR chloride methanolate crystal, and 3-carbamoyl-1-((2S,3R,4S,5R)-3,4-dihydroxy-5(hydroxymethyl)tetra hydrofuran-2-yl)pyridin-1-ium chloride. The first listed is more resistant to decomposition upon heating than other forms.

U.S. Pat. No. 8,106,184 describes the following NR analogues with reduced potential toxicities: O-ethyl NR (OENR), tri-O-acetyl O′-ethyl NR (TAENR), N-dimethyl NR (DMNR), and N-allyl NR (ANR).

Additional NR analogues are described in US20170189433 and U.S. Ser. No. 10/000,519. Examples include:

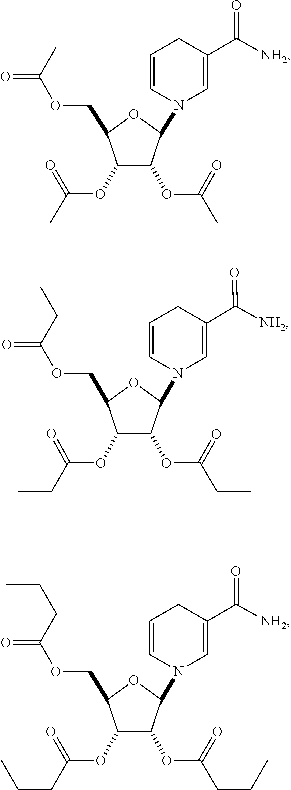 [SMILES: CC(=O)OC[C@H]1O[C@@H](N2C=CCC(C(N)=O)=C2)[C@H](OC(C)=O)[C@@H]1OC(C)=O ; CCC(=O)OC[C@H]1O[C@@H](N2C=CCC(C(N)=O)=C2)[C@H](OC(=O)CC)[C@@H]1OC(=O)CC ; CCCC(=O)OC[C@H]1O[C@@H](N2C=CCC(C(N)=O)=C2)[C@H](OC(=O)CCC)[C@@H]1OC(=O)CCC]

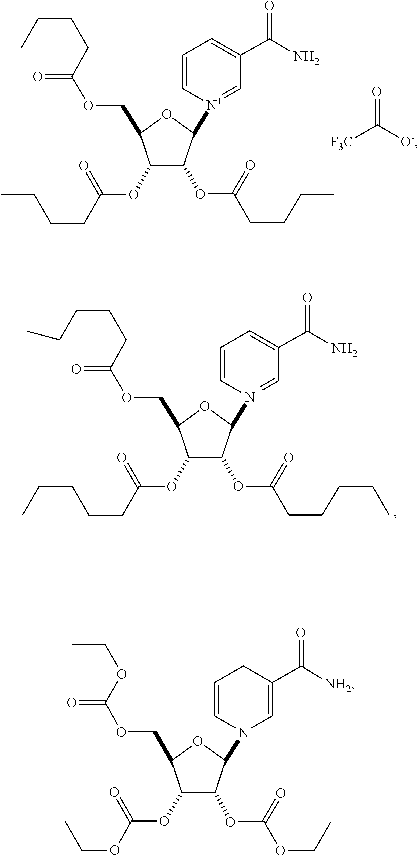

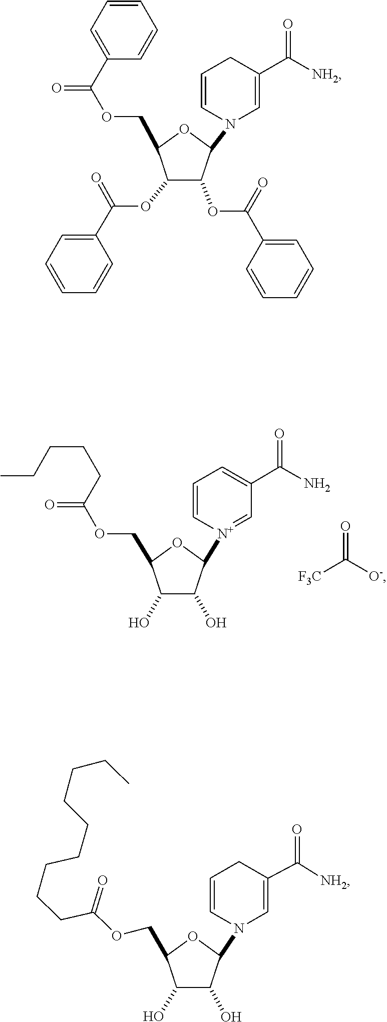

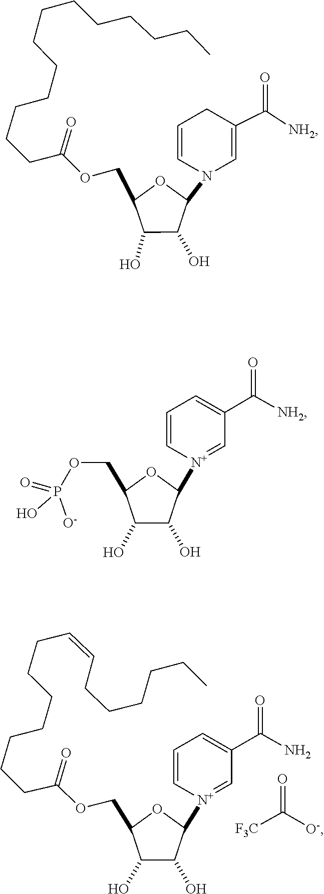

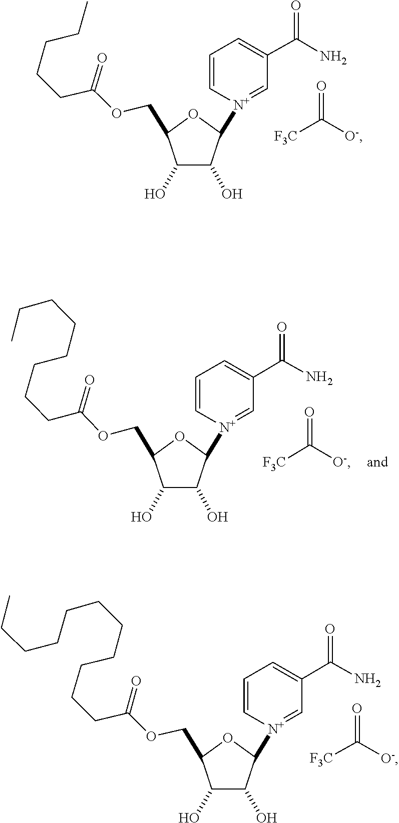

Additional exemplary NR analogues are described in, for example, Bioorg. Med. Chem. Lett. 2002, 12, 1135-1137; Franchetti et al., Bioorg. Med. Chem. Lett. 2004, 14, 4655-4658; and Yang, et al., Med. Chem. 2007, 50, 6458-6461.

In particular embodiments, NAD precursors include any compound including NR (e.g., NR chloride, NR bromide, OENR (O-ethyl nicotinamide riboside), TAENR (tri-O-acetyl O′-ethyl nicotinamide riboside), DMNR (N-dimethyl nicotinamide riboside), and ANR (N-allyl nicotinamide riboside)).

(iii) Compositions for Administration. NAD precursors can be formulated into compositions with a pharmaceutically acceptable carrier for administration to subjects. Salts and/or pro-drugs of NAD precursors can also be used.

A pharmaceutically acceptable salt includes any salt that retains the activity of the NAD precursor and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage of a NAD precursor or by hydrolysis of a biologically labile group.

Exemplary generally used pharmaceutically acceptable carriers include absorption delaying agents, antioxidants (e.g., ascorbic acid, methionine, vitamin E), binders, buffering agents, bulking agents or fillers, chelating agents (e.g., EDTA), coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA.

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the NAD precursor or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The formulations disclosed herein can be formulated for administration by, for example, oral administration. For oral administration, the compositions can be formulated as tablets, pills, dragees, capsules, liquids, gels, syrups, slurries, suspensions and the like. For oral solid formulations such as powders, capsules and tablets, suitable excipients include binders (gum tragacanth, acacia, cornstarch, gelatin), fillers such as sugars, e.g. lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

For injection, compositions can be formulated as aqueous solutions, such as in buffers including Hanks' solution, Ringer's solution, or physiological saline, or in culture media, such as Iscove's Modified Dulbecco's Medium (IMDM). Injectable compositions can be in lyophilized and/or powder form for constitution with a suitable vehicle, e.g., sterile pyrogen-free water, before use.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insufflator may also be formulated including a powder mix of active ingredients and a suitable powder base such as lactose or starch.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salt.

Additionally, compositions can be formulated as sustained-release systems utilizing semipermeable matrices of solid polymers including at least one NAD precursor. Various sustained-release materials have been established and are well known by those of ordinary skill in the art. Sustained-release systems may, depending on their chemical nature, release NAD precursor following administration for a few weeks up to over 100 days. Depot preparations can be administered by injection; parenteral injection; instillation; or implantation into soft tissues, a body cavity, or occasionally into a blood vessel with injection through fine needles.

Depot formulations can include a variety of bioerodible polymers including poly(lactide), poly(glycolide), poly(caprolactone) and poly(lactide)-co(glycolide) (PLG) of desirable lactide:glycolide ratios, average molecular weights, polydispersities, and terminal group chemistries. Blending different polymer types in different ratios using various grades can result in characteristics that borrow from each of the contributing polymers.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and compositions are disclosed in Remington's Pharmaceutical Sciences, 18th Ed. Mack Printing Company, 1990. Moreover, compositions can be prepared to meet sterility, pyrogenicity, general safety, and purity standards as required by U.S. FDA Office of Biological Standards and/or other relevant foreign regulatory agencies.

In particular embodiments, the compositions include NAD precursor of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Compositions disclosed herein can be formulated for administration by, for example, ingestion, injection, infusion, perfusion, or lavage. The compositions disclosed herein can further be formulated for oral, intravenous, intradermal, intraarterial, intranodal, intralymphatic, intraperitoneal, intralesional, intraprostatic, intravaginal, intrarectal, topical, intrathecal, intramuscular, intravesicular, and/or subcutaneous administration and more particularly by parenteral injection, including intravenous, intradermal, intraperitoneal, intramuscular, and/or subcutaneous injection.

(iv) Methods of Use. Methods disclosed herein include treating subjects (humans with cardiac dysfunction) with compositions disclosed herein. Treating subjects includes delivering therapeutically effective amounts. Therapeutically effective amounts include those that provide effective amounts, prophylactic treatments and/or therapeutic treatments. Therapeutically effective amounts within the current disclosure are high doses of NAD precursors.

An “effective amount” is the amount of a formulation necessary to result in a desired physiological change in the subject. For example, an effective amount can increase fractional shortening and reduce cardiac hypertrophy. For example, an effective amount can increase physiological markers such as upregulating mtDNA-encoded mRNA transcripts and UCPs; activating PPARα/RXRα; increasing SDR protein levels, such as Mrpp2; increasing RNAse P activity; and increasing OXPHOS protein and FAO protein. Effective amounts are often administered for research purposes. Effective amounts disclosed herein can cause a statistically-significant effect in an in vitro assay relevant to the assessment of cardiac dysfunction or a disorder associated with cardiac dysfunction. A composition can be administered in an effective amount, wherein the effective amount restores, at least in part, functionality of pathways known to be dysregulated in failing hearts.

A “prophylactic treatment” includes a treatment administered to a subject who does not display signs or symptoms of cardiac dysfunction or displays only early signs or symptoms of cardiac dysfunction such that treatment is administered for the purpose of diminishing or decreasing the risk of advancement of cardiac dysfunction. Thus, a prophylactic treatment functions as a preventative treatment against cardiac dysfunction.

A “therapeutic treatment” includes a treatment administered to a subject who displays symptoms or signs of cardiac dysfunction and is administered to the subject for the purpose of diminishing or eliminating those signs or symptoms of cardiac dysfunction. The therapeutic treatment can reduce, control, or eliminate the cardiac dysfunction and/or reduce, control or eliminate side effects of the cardiac dysfunction.

Function as an effective amount, as a prophylactic treatment, or as a therapeutic treatment are not mutually exclusive, and in particular embodiments, administered dosages may accomplish more than one such function.

Administration of the compositions described herein can increase physiological markers, such as those described above, by at least 10%, or at least 20%, or at least 25%, or at least 30%, or at least 35%, or at least 40%, or at least 45%, or at least 50%, or at least 55%, or at least 60%, or at least 65%, or at least 70%, or at least 80%, at least 90%, at least 92%, at least 95%, at least 97%, at least 98%, or at least 99%.

The treatment of cardiac dysfunction can refer to the statistically significant reduction of at least one symptom of cardiac dysfunction within a 90% confidence interval, where a symptom may be one of dyspnea, fatigue, pulmonary congestion, chest pain, other bodily pain, and the like. Any statistically significant attenuation of such one or more symptoms of cardiac dysfunction is considered to be a treatment thereof. In some embodiments, compositions are administered, the compositions including dosages of an amount of at least one NAD precursor as described herein, effective to treat or prevent the clinical symptoms of cardiac dysfunction to a statistically significant extent within a 90% confidence interval.

For administration, therapeutically effective amounts (also referred to herein as doses) are high dose regimens of NAD precursors. The actual dose and administration protocol for a particular subject can be determined by a physician taking into account parameters such as physical and physiological factors including target, body weight, severity of condition, type of condition, previous or concurrent therapeutic interventions, idiopathy of the subject and route of administration

The Experimental Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (v) Experimental Example 1

Methods. Nicotinamide riboside chloride (NR) was supplied as powder by the manufacturer (Niagen®, ChromaDex). Stock solutions (50 mg/ml) were made in 0.9% saline for intraperitoneal (IP) administration.

All procedures involving animal use were approved by the Institutional Animal Care and Use Committee (IACUC) of the University of Washington. Wild-type mice (129S1/SvlmJ Stock No: 002448) and Sirtuin 3 knockout mice (129-Sirt3tm1.1Fwa/J Stock No: 012755) were purchased from Jackson Laboratories. All mice were housed at 22° C. with a 12-hr light, 12-hr dark cycle with free access to water and standard chow. Male mice were used for experiments in this study.

Transverse aortic constriction. Male mice (3-4 months old) underwent transverse aortic constriction (TAC) or sham surgery. Briefly, mice were anesthetized with an IP injection of 130 mg/kg ketamine and 8.8 mg/kg xylazine in saline. Mice were intubated with 20 G cannula and ventilated 140 breaths per minute by small animal TOPO ventilator (Kent Scientific). The aortic arch was exposed via a left thoracotomy and by carefully separating the thymus. A constriction of the transverse aorta was generated by tying a 6-0 Ethilon ligature against a 27-gauge blunt needle around the aorta between the brachiocephalic and left common carotid arteries. Promptly the needle was removed and the chest and skin were closed by 5-0 polypropylene suture. The animal was removed from ventilation and kept on a heating blanket during recovery from anesthesia. SR buprenorphine (0.05 mg/kg) was administered subcutaneously for analgesia. Sham operated mice underwent all the same procedures as TAC mice excluding the constriction of the aorta. All mice were monitored every 12 h during the first 72 h post-surgery, followed by daily visits over the next 4 weeks.

Minipump implantation. Pathologic hypertrophy was induced by treating male (3-4 months old) Sirt3ko and WT mice with isoproterenol (ISO) or vehicle for 14 days. Mice with and without GW6471(4.5 mg/kg/day) were treated with NR (500 mg/kg body weight IP daily) or vehicle starting at day one after mini-pump insertion. Briefly, an incision was made in the skin between the shoulder blades. A deep subcutaneous pocket was made caudal to the incision using blunt dissection. The sterile minipump (Alzet #2002) was inserted into the pocket, and the incision was closed with wound clips. Isoproterenol (30 mg/kg/day) was delivered by osmotic minipump for 14 days. Minipumps carrying saline were used as a control.

Transthoracic Echocardiography. Echocardiography of hearts was performed at indicated time after surgery using the VEVO 2100 high-frequency, high-resolution digital imaging system (VisualSonics) equipped with a MS400 Microscan Transducer on anesthetized mice (0.5%-2% isoflurane in 95% oxygen). Measurements were made when heart rate was >450 bpm. Cardiac function and geometry measurements were measured in parasternal short axis view. M-mode images were used for analysis and calculated by the average of at least three cardiac cycles and carried out in a blind fashion. Hearts were harvested at 12-week post-surgery for subsequent experiments and biochemical assays.

Western blot. For Western blot procedures cardiac tissues were homogenized with RIPA buffer (ThermoScientific Pierce RIPA Buffer #89900) and protease inhibitors (Roche Cat. No. 11-836-170-001) using bullet blender at 4° C. for 10 minutes. Protein concentrations of supernatants were collected after centrifugation and quantified using Pierce™ BCA® Protein Assay Kits and Reagents (#23225). Protein lysates in Lammeli sample buffer were loaded to 10% SDS-PAGE, transferred to PVDF membrane and blocked with 5% BSA in TSBT. Specific proteins were detected by specific antibodies listed above and corresponding secondary antibodies. Signals were visualized by HRP derived chemiluminescence (Amersham ECL Western Blotting Detection System #RPN2106) and film. Protein levels were quantified by Image-J.

NAD⁺/NADH determination. NAD⁺ and NADH were determined in 8 mg heart tissue using BioAssay Systems EnzyChrom NAD⁺/NAD Assay Kit (E2ND-100) per manufactures instructions.

RNA Extraction and Quantitative Real-Time Polymerase Chain Reaction (RT-PCR). Total RNA was isolated from frozen heart tissue using the RNeasy fibrous Kit (Qiagen). Total RNA was reverse-transcribed into first-strand cDNA using Iscript (Bio-rad) according to manufacturer's guidelines. Real Time PCR was performed using SYBR green (Bio-Rad). Results of mRNA levels were normalized to 18S rRNA levels and reported as fold-change over control. The primer information is listed in Table 2 below.

| | |
|---|---|
| Mouse natriuretic peptide type A (Nppa) | GCTTCCAGGCCATATTGGAG (SEQ ID |
| forward | NO: 1) |
| Mouse natriuretic peptide type A (Nppa) | GGGGGCATGACCTCATCTT (SEQ ID |
| reverse | NO: 2) |
| Uncoupling protein 3 (UCP3) forward | TGCTGAGATGGTGACCTACGA (SEQ |
| | ID NO: 3) |
| Uncoupling protein 3 (UCP3) reverse | CCAAAGGCAGAGACAAAGTGA (SEQ |
| | ID NO: 4) |
| Uncoupling protein 2, mitochondrial proton | GCGTTCTGGGTACCATCCTA (SEQ ID |
| carrier (UCP2) forward | NO: 5) |
| Uncoupling protein 2, mitochondrial proton | GCTCTGAGCCCTTGGTGTAG (SEQ ID |
| carrier (UCP2) reverse | NO: 6) |
| Medium chain acetyl-Coenzyme A | AGGGTTTAGTTTTGAGTTGACGG |
| dehydrogenase (MCAD) forward | (SEQ ID NO: 7) |
| Medium chain acetyl-Coenzyme A | CCCCGCTTTTGTCATATTCCG (SEQ ID |
| dehydrogenase (MCAD) reverse | NO: 8) |
| Peroxisome proliferator activated receptor | CATACTCGCGGGAAAGACCA (SEQ ID |
| alpha (PPARA) forward | NO: 9) |
| Peroxisome proliferator activated receptor | CGTCTTCTCGGCCATACACA (SEQ ID |
| alpha (PPARA) reverse | NO: 10) |
| CPT1b forward | TCTAGGCAATGCCGTTCAC (SEQ ID |
| | NO: 11) |
| CPT1b reverse | GAGCACATGGGCACCATAC (SEQ ID |
| | NO: 12) |
| Ubiquinol-cytochrome c reductase core | AGACCCAGGTCAGCATCTTG (SEQ ID |
| protein 1 (Uqcrc1) forward | NO: 13) |
| Ubiquinol-cytochrome c reductase core | GCCGATTCTTTGTTCCCTTGA (SEQ ID |
| protein 1 (Uqcrc1) reverse | NO: 14) |
| MT-ND1 mitochondrially encoded NADH | GGATCCGAGCATCTTATCCA (SEQ ID |
| dehydrogenase 1 forward | NO: 15) |
| MT-ND1 mitochondrially encoded NADH | GGTGGTACTCCCGCTGTAAA (SEQ ID |
| dehydrogenase 1 reverse | NO: 16) |
| cox5b forward (nuclear coded) | GTGTCCCCACTGATGAGGAG (SEQ ID |
| | NO: 17) |
| cox5b reverse (nuclear coded) | TATGGGTCCAGTCCCTTCTG (SEQ ID |
| | NO: 18) |
| CytB forward | ATTCCTTCATGTCGGACGAG (SEQ ID |
| | NO: 19) |
| CytB reverse | ACTGAGAAGCCCCCTCAAAT (SEQ ID |
| | NO: 20) |
| Transcription factor A, mitochondrial | CCCGGCAGAGACGGTTAAAA (SEQ ID |
| (TFAM) forward | NO: 21) |
| Transcription factor A, mitochondrial | TCCCTGAGCCGAATCATCCT (SEQ ID |
| (TFAM) reverse | NO: 22) |
| estrogen related receptor, alpha (Esrra) | CCAGGCTTCTCCTCACTGTC (SEQ ID |
| forward | NO: 23) |
| estrogen related receptor, alpha (Esrra) | GCCCCCTCTTCATCTAGGAC (SEQ ID |
| reverse | NO: 24) |
| Peroxisome proliferator activated receptor | GTAAATCTGCGGGATGATGG (SEQ ID |
| gamma co-activator 1 alpha (PGC1A) | NO: 25) |
| forward | |
| Peroxisome proliferator activated receptor | AGCAGGGTCAAAATCGTCTG (SEQ ID |
| gamma co-activator 1 alpha (PGC1A) | NO: 26) |
| reverse | |
| connective tissue growth factor (ctgf) | AGAACTGTGTACGGAGCGTG (SEQ ID |
| forward | NO: 27) |
| connective tissue growth factor (ctgf) | GTGCACCATCTTTGGCAGTG (SEQ ID |
| reverse | NO: 28) |
| Hsd17b10(f) | GGATGTACCTGACTCAGAGGGT (SEQ |
| | ID NO: 29) |
| Hsd17b10(r) | TACCTGCACAGTTGACAGCCAC (SEQ |
| | ID NO: 30) |
| Mrpp1(f) | CATAGCAACAGAGTGCCTTCCAC |
| | (SEQ ID NO: 31) |
| Mrpp1(r) | CCTGGGAACAAACTTGAGAGCC (SEQ |
| | ID NO: 32) |
| slc22a5 (Octn2)-forward | ACGATGGCTCATCTCTCAAGGC (SEQ |
| | ID NO: 33) |
| slc22a5 (Octn2)-reverse | TGTGGTGCAACTGAGGCTTCGT (SEQ |
| | ID NO: 34) |
| Rdh11 forward | AGTCACTGGTGCTAACACAGGC (SEQ |
| | ID NO: 35) |
| Rdh11 reverse | TGTTCCCTGTGACGGCTTGGAT (SEQ |
| | ID NO: 36) |
| Rdh7 forward | CCTGGTCAACAATGCTGGCATC (SEQ |
| | ID NO: 37) |
| Rdh7 reverse | CATGCTCAGAGTCACCTCGATC (SEQ |
| | ID NO: 38) |
| Rdh14 forward | GCTTTCTGCCAAGAACTGCTACA |
| | (SEQ ID NO: 39) |
| Rdh14 reverse | CCAGATGGTTCACTCCAAACTGC |
| | (SEQ ID NO: 40) |
| mouse 18S forward | GGACAGGATTGACAGATTGATAG |
| | (SEQ ID NO: 41) |
| mouse 18S reverse | ATCGCTCCACCAACTAAGAA (SEQ ID |
| | NO: 42) |
| Rxra forward | GGCTTCGGGACTGGTAGCC (SEQ ID |
| | NO: 43) |
| Rxra reverse | GCGGCTTGATATCCTCAGTG (SEQ ID |
| | NO: 44) |
| Atp5a1 forward | ACCAGGATTTGTGTGGTGGTCAGA |
| | (SEQ ID NO: 45) |
| Atp5a1 reverse | AGCCATTGCTGAGGTCACACAGTA |
| | (SEQ ID NO: 46) |
| Nd3 forward | CCATATGAATGCGGATTCGACCCT |
| | (SEQ ID NO: 47) |
| Nd3 reverse | GCTCATGGTAGTGGAAGTAGAAGAGC |
| | (SEQ ID NO: 48) |
| Pparg forward | GGAAGACCACTCGCATTCCTT (SEQ |
| | ID NO: 49) |
| Pparg reverse | TCGCACTTTGGTATTCTTGGAG (SEQ |
| | ID NO: 50) |

The mitochondrial junctional RNA primers are listed in Table 3 below.

| | |
|---|---|
| ND6 forward | GCTAACCCAAGACAACCAACC |
| | (SEQ ID NO: 51) |
| CYTB reverse | CAGACTCCTAGAAGGGACCCA |
| | (SEQ ID NO: 52) |
| ND1 forward | GCGGGAGTACCACCATACA (SEQ |
| | ID NO: 53) |
| ND2 reverse | GATGATGGCAAGGGTGATAGGA |
| | (SEQ ID NO: 54) |
| COX3 forward | GTCTGACTTTTCCTATACGTCTCCA |
| | (SEQ ID NO: 55) |
| ND3 reverse | TGGGGGAGTCAGAATGCAAC (SEQ |
| | ID NO: 56) |
| COX1 forward | ACACATTCGAGGAACCAACCT |
| | (SEQ ID NO: 57) |
| COX2 reverse | GGGGATGTGGCGTCTTGTAG (SEQ |
| | ID NO: 58) |
| 12s-Val-16s forward | GTGCTTGGAATAATCATAGTGTAGC |
| | (SEQ ID NO: 59) |
| 12s-Val-16s reverse | GGGCTAGGATTAGTTCAGAGTGT |
| | (SEQ ID NO: 60) |

Cardiac acyl-carnitines. Metabolites were extracted from frozen heart tissue (10 mg) by precipitating in 80:20% methanol:water following the addition of 1 ug/ml internal standard (C16 palmitoylcarnitine, d3, Cambridge Isotope Laboratories, Inc.). After centrifugation, the supernatant was transferred to LC/MS auto sampler vials (Thermo Fisher). The protein pellet was redissolved in 0.1 M NaOH and protein concentration measured for normalization.

Acyl-carnitines were analyzed using a Triple TOF—5600 (AB Sciex) series mass spectrometer in line with a Water Acquity I-class UPLC. Chromatographic separation was achieved using an Acquity UPLC BEH HILIC, 1.7 micron, 2.1×100 mm column (Waters) with a flow rate of 0.5 ml/min. Total run time was 22 minutes. The mobile phase was composed of two solvents: A=5 mM Ammonium Acetate in 95:5 acetonitrile:water and B=5 mM Ammonium Acetate in 50:50 acetonitrile:water using the following gradient.

| Time (min) | % B |
|---|---|
| 0.00 | 0.0 |
| 10.0 | 0.0 |
| 13.0 | 50.0 |
| 14.0 | 50.0 |
| 14.5 | 0.0 |

Samples were analyzed in positive electrospray ionization (ESI+) with a mass range from 50-1000 Da. Mass spectrometer parameters were as follows: Source Temperature (TEM)—500° C., Declustering Potential (DP)—70, Curtain Gas (CUR)—30, Collision Energy (CE)—45, Ion Source Gas 1 (GS1)—45, Ion Source Gas 2 (GS2)—40. Peaks were integrated using MultiQuant 3.0.2 software (AB Sciex 2015). Mass-to-charge ratio (m/z) and retention time (RT) of individual metabolites were annotated according to known standards (Cambridge Isotope Laboratories). Additional acyl-carnitines were identified by their m/z value as compared to theoretical values determined by the molecular formula ±0.003 D. Peak areas intensities were normalized to protein concentration.

Retinoid measurements. Analysis of all-trans-retinyl ester palmitate (RP), all-trans,-retinol (ROL) was done in collaboration with Dr. Nina Isoherranen laboratory using previously established methods (Czuba et al., 2020). RP, ROL were purchased from Toronto Research Chemicals (North York, ON), and retinyl ester palmitate-d4 (RP-d4) and all-trans-retinol-d6 (ROLd6) were purchased from Cambridge Isotopes (Andover, MA). Blank human serum (DC MASS SPECT GOLD MSG4000) for standard curve and quality control matrix was purchased from Golden West Diagnostics (Temecula, CA). Solvents for liquid chromatography tandem mass spectrometry (LC-MS/MS) were Optima LC-MS grade and purchased from Fisher Scientific (Pittsburgh, PA).

Heart tissue samples (25-40 mg) were processed under yellow light, on ice to prevent degradation and isomerization of the retinoids. Samples were diluted with 5× volume of 0.9% saline on ice, 20 μL of internal standard mix in methanol containing 2 μM conc of RP-d4 and 20 μM of ROL-d6 was added before homogenization in an Omni Bead Ruptor 24 Bead Mill homogenizer (Omni International, Inc., Kennesaw, GA) with ceramic beads at 4° C. Following homogenization, samples were vortexed and transferred via Pasteur pipette to glass culture tubes, vortexed with ethanol containing 0.025 M KOH, followed by extraction with 10 mL hexanes. After centrifugation at 480×g for 5 min, the organic hexane layer was transferred and evaporated at 37° C. under nitrogen. The residue was reconstituted in 90 μL of acetonitrile and transferred to amber vials with glass inserts for LC-MS/MS analysis.

RP and ROL concentrations were determined on an AB Sciex 5500 QTRAP mass spectrometer (AB Sciex, Foster City), coupled with an Agilent 1290 UHPLC (Agilent, Santa Clara, CA) using a Ascentis Express RP-amide column (2.1×150 mm, 271 μm) and guard column (2.1×5 mm, 2.7 μm). The autosampler was set at 6° C., the injection volume was 5 μL the column oven was set to 40° C. The gradient was water with 0.1% formic acid (A) and acetonitrile with 0.1% formic acid (B), with flow rate at 0.5 mL/min at 60% B held for 2 min, increasing to 66% B at 9.2 min, then increasing to 100% B at 13 min, held at 100% B until 23 min before returning to initial conditions for 4 min.

RP and ROL were detected in positive ion mode atmospheric pressure chemical ionization (APCI) mode using selected reaction monitoring (SRM). The parameters were as follows: TEM 350° C., CUR 35, and GS1 80. The mass transitions were 292.2>93.0 for RP and ROL, 275.2>96.0 for ROL-d6, and 273.2>94.0 for RP-d4. The CE was 25 V, DP was 60 V, entrance potential was 10 V, and collision exit potential was 10 V. Peaks were integrated and quantified using MultiQuant 2.1.1 (AB Sciex, Foster City, CA). Standard curves were weighted 1/x and linear over the range 0.78-400 pmol for RP and ROL, with ⅔ of quality control samples within 15% of nominal concentration.

Targeted Metabolomics. Analysis of metabolites was done by The Northwest Metabolomics Research Center at the University of Washington. Briefly, aqueous metabolites for targeted LC-MS profiling of heart tissue samples were extracted from frozen heart tissue (10 mg) by precipitating in 80:20% methanol:water. After centrifugation, the supernatant was transferred to LC/MS auto sampler vials (Thermo Fisher). The protein pellet was redissolved in 0.1M NaOH and protein concentration measured for normalization. Samples were first homogenized in 200 μL purified deionized water at 4° C., and then 800 μL of cold methanol. Afterwards samples were vortexed, stored for 30 minutes at −20° C., sonicated in an ice bath for 10 minutes, centrifuged for 15 min at 14,000 rpm and 4° C., and then 600 μL of supernatant was collected from each sample. Lastly, recovered supernatants were dried on a SpeedVac and reconstituted in 0.2 mL of LC-matching solvent containing 17.8 μM 2C13-tyrosine and 39.2 3C13-lactate (reference internal standards were added to the reconstituting solvent in order to monitor LC-MS performance). Samples were transferred into LC vials and placed into a temperature controlled autosampler for LC-MS analysis.

LC-MS Assay. Targeted LC-MS metabolite analysis was performed on a duplex-LC-MS system composed of two Shimadzu UPLC pumps, CTC Analytics PAL HTC-xt temperature-controlled auto-sampler (set at 4° C.) and AB Sciex 6500+ Triple Quadrupole MS equipped with ESI ionization source (2). UPLC pumps were connected to the autosampler in parallel and were able to perform two chromatography separations independently from each other. Each sample was injected twice on two identical analytical columns (Waters XBridge BEH Amide XP) performing separations in hydrophilic interaction liquid chromatography (HILIC) mode. While one column was performing separation and MS data acquisition in ESI+ ionization mode, the other column was getting equilibrated for sample injection, chromatography separation and MS data acquisition in ESI-mode. Each chromatography separation was 18 minutes (total analysis time per sample was 36 minutes). MS data acquisition was performed in multiple-reaction-monitoring (MRM) mode. LC-MS system was controlled using AB Sciex Analyst 1.6.3 software. Measured MS peaks were integrated using AB Sciex MultiQuant 3.0.3 software. The LC-MS assay was targeting 361 metabolites (plus 2 spiked reference internal standards). Up to 192 metabolites (plus 2 spiked standards) were measured across the study set, and over 95% of measured metabolites were measured across all the samples. In the addition to the study samples, two sets of quality control (QC) samples were used to monitor the assay performance as well as data reproducibility. One QC [QC(I)] was a pooled human serum sample used to monitor system performance and the other QC [QC(S)] was pooled study samples and this QC was used to monitor data reproducibility. Each QC sample was injected per every 10 study samples. The data were well reproducible with a median CV of 7.0%.

RNA Sequencing. Total RNA was extracted from 30 mg of heart tissue using the Qiagen RNeasy kit. RNA Sequencing and quality control was performed BGI (BGISEQ-500). Data Analysis-RNAseq samples were aligned using Tophat (Trapnell et al., 2009) (version 2.0.13). Genelevel read counts were quantified using htseq-count (Anders et al., 2015). Genes expressed above 4TPM in at least 2 samples were used. In differential expression (DESeq), A gene to have total normalized reads above 10 across all samples. DeSeq (Anders and Huber, 2010) was used for differential gene expression analysis. Genes with fold change >1.5 and FDR <0.1 were considered differentially expressed. GSEA was performed using Broad Institute software against the Reactome database. Iregulon v1.3 was used to predict transcription factor binding motif. The following options were used: Motif collection 10 K (9713 PWMs), putative regulatory region 20 kb centered around TSS (7 species). Program default settings for Recovery and Transcription factor prediction options were selected for the analysis.

Mitochondrial Isolation from cardiac tissue. Hearts were excised from mice, the aortas and atria were removed. Heart tissues were rinsed briefly in ice-cold mitochondria isolation medium (MIM) (70 mM sucrose, 220 mM mannitol, 5 mM MOPS, 1 mM EDTA, 0.025% fatty acid-free BSA, pH 7.4 with 5M KOH) to remove residual blood. Tissues were minced on ice and resuspended in fresh MIM, followed by trypsin digestion (10 ug/ml) and incubated on ice for 10 min. Trypsin digestion was stopped by the addition of trypsin inhibitor (0.5 mg/ml) and additional BSA (1 mg/ml) to MIM. The tissue pellets were resuspended in fresh MIM containing 1 mg/ml BSA, and transferred to a Teflon-glass tube and homogenized on ice with a Teflon pestle. The homogenates were centrifuged for 10 min at 800×g at 4° C. The supernatants were collected and centrifuged for 10 min at 8,000×g at 4° C. The supernatant was discarded and the mitochondrial pellets were resuspended in MIM to wash. The resuspension was centrifuged for 10 min at 8,000×g at 4° C., and the supernatant discarded. The mitochondrial pellet was resuspended to a concentrated volume for protein quantification by BCA Assay (Pierce).

Organ weight and histological assessment. Hearts from mice were harvested, rinsed briefly in 1×PBS, blotted dry, and weighed. Lungs from the same mice were harvested, weighed, then dried at 56 C. After 72 hours the lungs were weighed again. Heart weight (HW) and Lung weight (LW) was normalized to tibia length (TL) to assess changes in hypertrophic growth and lung edema, respectively. For histology, hearts were perfusion fixed with 10% neutral buffered formalin. Longitudinal sections of hearts were stained with Masson's Trichrome for assessment of fibrosis by the University of Washington Histology and Imaging Core.

Mitochondrial respirations. Isolated mitochondria respiration experiments were performed in an Clark type oxygen electrode Oxytherm System (Hansatech Instruments) at 30C. Briefly, freshly isolated mitochondria were added to respiration buffer (125 mM KCl, 20 mM HEPES, 3 mM MgCl2, 400 μM EGTA, 5 mM KH2PO4, 0.2% BSA (fatty acid free), pH 7.2) and placed in the respiration chamber to measure baseline respiration (State 1). The change in oxygen levels following the immediate addition of substrates (pyruvate/malate (5 mM/2 mM) or palmitoylcarnitine/malate (50 μM/2 mM) (State 2) followed by the sequential addition of 2.5 mM ADP (State 3), 1 mg/ml Oligomycin (State 4p) and 1 μM FCCP (FCCP Max). Antimycin A (2.5 μM) was added last to measure the residual oxygen consumption not accounted for by the electron transfer chain and it was subtracted from the previous measurements.

Enzyme Activities. Citrate synthase (CS), Complex I (CI) and Cytochrome C Oxidase (CIV), and Mrpp2 dehydrogenase activities were determined in heart tissue homogenates at 30° C. using a protocol adapted from previously published methods (Spinazzi et al., 2012). Briefly, for CS activity frozen cardiac tissue (25 mg) was homogenized at 4° C. in CelLytic™ MT Cell Lysis Reagent (C3228) and then incubated on ice for 30 minutes. The samples were then centrifuged at 10,000×g for 10 minutes at 4° C. and protein concentration determined. CS activity was analyzed in 100 mM Tris buffer containing 0.1% Triton X-100, pH 8.0 as previously described (Spinazzi et al., 2012). For CIV activity, 25 mg of cardiac tissue was homogenized at 4° C. in 50 mM potassium phosphate buffer containing 1 mM EDTA, and 0.1% Triton X-100 (pH 7.4; final concentration, 5 mg tissue/mL). The samples were incubated on ice for 30 minutes followed by centrifugation at 10,000×g for 10 minutes at 4° C. CIV activity was analyzed in 100 mM potassium phosphate buffer as previously described (Spinazzi et al., 2012). For Cl activity, 25 mg of cardiac tissue was homogenized at 4° C. in 50 mM potassium phosphate buffer containing 1 mM EDTA, and 0.1% Triton X-100 (pH 7.4; final concentration, 5 mg tissue/mL). The samples were incubated on ice for 30 minutes followed by centrifugation at 10,000×g for 10 minutes at 4° C. Cl activity was analyzed in 100 mM potassium phosphate buffer as previously described (Spinazzi et al., 2012). In parallel, a separate reactions containing the same quantity of reagents and sample but with the addition of 10 μl of 1 mM rotenone solution. The reactions were started by adding 6 μl of ubiquinone (10 mM), mix and follow the decrease of absorbance at 340 nm for 2 min. The dehydrogenase activity of Mrpp2 was measured as described (Falk et al., 2016) in the reverse direction for reduction of acetoacetyl-CoA (30 mM) to L-3-hydroxyacyl-CoA. The reaction was monitored as the decrease of absorbance at 340 nm.

Chromatin immunoprecipitation (ChIP). The EZ ChIP kit (EMD Millipore catalog #17-371) was used according to the manufacturer's protocol. Briefly, heart tissue was cut into small pieces using a razor blade in a petri dish on ice. The cubed tissue was incubated in fixation solution (1% formaldehye) in a conical tube and rotated at room temperature for 15 minutes to crosslink proteins and chromatin. The reaction was stopped by adding 0.125M Glycine for 5 min at room temperature. The contents were homogenized with a hand-held tissue homogenizer for 45 seconds then pelleted by centrifugation for 3 minutes at 1,250×g at 4C. The supernatant was discarded and the pellet was resuspended in 10 ml ice-cold PBS wash buffer followed by centrifugation at 1,250×g for 3 minutes. This was wash step was repeated on ice. The supernatant was discarded and the pellet resuspended in 5 ml chromatin prep buffer and incubated on ice for 10 minutes. The resuspended pellet was homogenized on ice with a dounce homogenizer for 30 strokes and then contents transferred to new conical tube on ice. The suspension was centrifuged 3 minutes at 1,250×g at 4C then supernatant discarded. The pellet was resuspended in 1 ml Chip buffer and transferred to new centrifuge tube then incubated on ice for 10 minutes. The contents were then subjected to sonication 5 times for 30 s with 30 s intervals. Purified chromatin was analyzed on a 1% agarose gel to determine the shearing efficiency. As a control, normal rabbit IgG (Cell Signaling #2729) was used as a replacement for the RXRα rabbit antibody (Cell Signaling D6H10 #3085). The ChIP procedure was performed as in the supplier's protocol (EZ-ChIP EMD Millipore catalog #17-371)). The Acadm primers for CHIP described here (Oka et al., 2015) with the following sequences:

(SEQ ID NO: 61)
5′-ATCTAGCCCAGAATTTGTTGTTCCAGTG-3′
and
(SEQ ID NO: 62)
5′-TCTAGGCCAGAGGGCGCAG-3′.

Antibodies. Acetylated lysine antibody (Cell Signaling 9441s), Mrpp2 (Hsd17b10, ERAB) from Proteintech catalog number 10648-1-AP, Mrrp1 (Trmt10c) from Proteintech catalog number 29087-1-AP, Mt-C01 from Abcam [1D6E1A8] (ab14705), and Mt-Nd1 from Santa Cruz (C-18) catalog sc-20493, RXRα (Cell Signaling D6H10) Rabbit mAb, SirT3 (Cell Signaling D22A3) Rabbit mAb, ATP5B (E-1) Santa Cruz Biotechnology sc-55597.

Statistics. All data presented as mean±standard error of the mean (SEM). Statistical analysis was performed with GraphPad Prism 8.3 (GraphPad Software, San Diego, CA). Normal distribution of the data was analyzed using a Shapiro-Wilk test. Comparisons between 3 or more groups were conducted by one-way or two-way ANOVA followed by a Tukey post-hoc analysis. For repeated measurements of multiple groups, mixed-effects analysis was performed. Comparisons between 2 groups was done by unpaired, two tailed t-test. All results were tested at the P<0.05 level of significance. For correlation graphs linear regression analysis was run on GraphPad Prism 8.3.

### (vi) Experimental Example 2

Mice 2-4 months old were used, weighing 25 to 27 g, with water intake ranging from 3.5 to 6.5 ml/day.

To each mouse, either NR as an NAD precursor was administered daily or vehicle was administered daily, for up to 10 days, either by IP injection or orally by drinking water. Saline was used as vehicle for IP injection. For oral administration, vehicle was unmodified drinking water.

NR was supplied substantially as in Experimental Example 1. For IP administration, NR was diluted in sterile, pharmaceutical-grade isotonic saline at concentrations based on weight-based dosing per subject to provide respective 500 mg/kg body weight daily doses as administrated by IP injection.

Additionally, for oral administration, NR was dissolved in sterile filtered water at concentrations based on weight-based dosing per subject to provide respective 400 mg/kg body weight daily doses, provided for drinking adlibitum. Generally, a mouse will drink ˜6 mL of water per day, equivalent to 7.7 ml/30 g of body weight (Bachmanov, et. al. Behavior Genetics, 2002, 32(6):435-443). All drinking water was replaced at least once a week with newly prepared NR solutions or vehicle. Over the first several days of oral administration of NR solutions, the consumption of NR water was monitored daily to verify that mice are not finding the NR water unpalatable and becoming dehydrated.

After 0 days; after 3 or 7 days of oral NR administration; and after 10 days of NR administration by IP injection, hearts and livers were harvested for biochemical assays. NAD⁺ and NADH were determined in approximately 6 mg heart tissue and approximately 6 mg liver tissue using BioAssay Systems EnzyChrom NAD⁺/NAD Assay Kit (E2ND-100) per manufactures instructions.

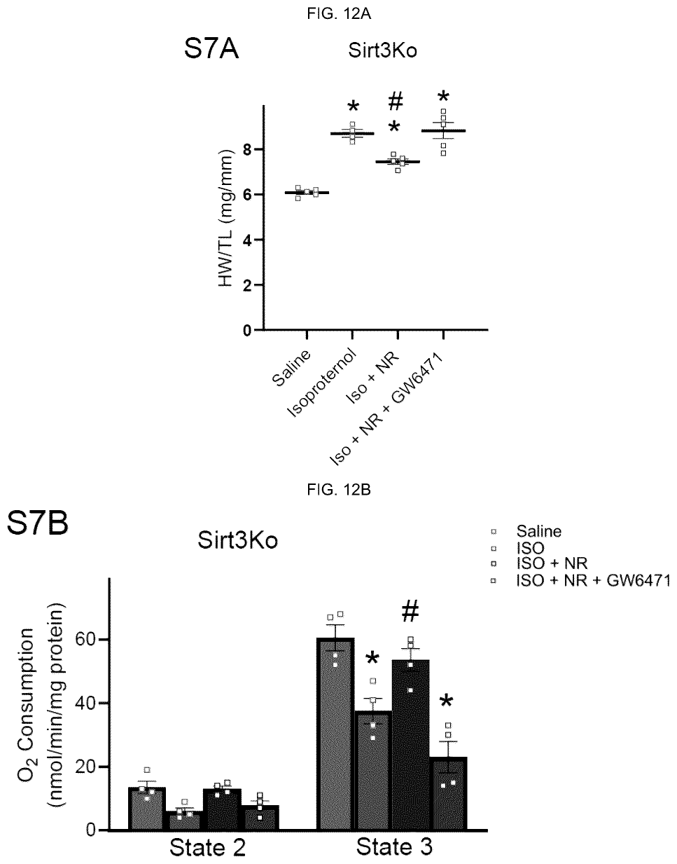

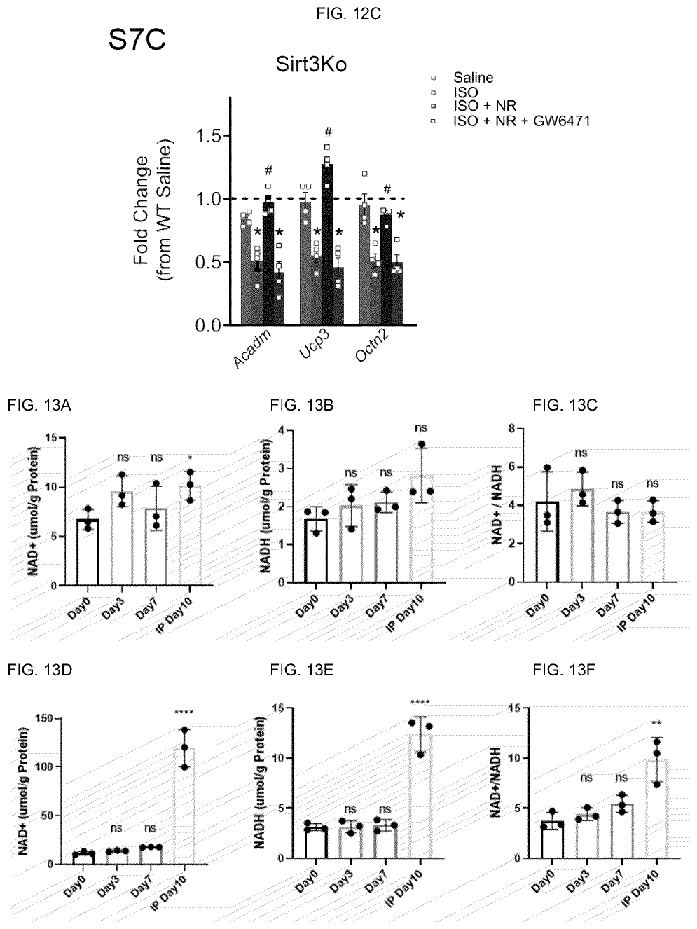

NR treatments increased cardiac levels of NAD(H) in hearts and livers (FIGS. 13A-13F). Furthermore, greater increases were observed in hearts and livers by IP injection NR treatments over orally administered NR treatments.

(vii) Closing Paragraphs. Unless otherwise indicated, the practice of the present disclosure can employ conventional techniques of chemistry, organic chemistry, biochemistry, analytical chemistry, and physical chemistry. These methods are described in the following publications. See, e.g., Harcourt, et al., Holt McDougal Modern Chemistry: Student Edition (2018); J. Karty, Organic Chemistry Principles and Mechanisms (2014); Nelson, et al., Lehninger Principles of Biochemistry 5th edition (2008); Skoog, et al., Fundamentals of Analytical Chemistry (8th Edition); Atkins, et al., Atkins' Physical Chemistry (11th Edition).

Each embodiment disclosed herein can comprise, consist essentially of, or consist of its particular stated element, step, ingredient, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e., denoting somewhat more or somewhat less than the stated value or range, to within a range of ±20% of the stated value; 19% of the stated value; ±18% of the stated value; 17% of the stated value; 16% of the stated value; ±15% of the stated value; 14% of the stated value; ±13% of the stated value; 12% of the stated value; 11% of the stated value; 10% of the stated value; 9% of the stated value; 8% of the stated value; 7% of the stated value; ±6% of the stated value; 5% of the stated value; 4% of the stated value; ±3% of the stated value; 2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified, thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when the application of the meaning renders any construction meaningless or essentially meaningless. In cases where the construction of the term would render it meaningless or essentially meaningless, the definition should be taken from Webster's Dictionary, 3rd Edition, or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method of treating cardiac dysfunction in a subject in need thereof, comprising:
parenterally administering a therapeutically effective amount of a nicotinamide adenine dinucleotide (NAD) precursor to the subject effective to stimulate intracellular oxidized nicotinamide adenine dinucleotide (NAD⁺) synthesis while bypassing nicotinamide phosphoribosyltransferase (NAMPT);
wherein administering the therapeutically effective amount increases a physiological marker not associated with Sirt3-mediated protein deacetylation in myocardial tissue of the human subject.

2. The method of claim 1, wherein the NAD precursor is nicotinamide riboside chloride (NR).

3. The method of claim 1, wherein the method further comprises treating ventricular cavity dilation in the subject.

4. The method of claim 1, wherein the method further comprises treating lung edema in the subject.

5. The method of claim 1, wherein the physiological marker comprises mitochondrial DNA (mtDNA) encoded mRNA.

6. The method of claim 1, wherein the physiological marker comprises an uncoupling protein (UCP).

7. The method of claim 1, wherein the physiological marker comprises PPARα/RXRα expression.

8. The method of claim 1, wherein the physiological marker comprises short-chain dehydrogenase/reductases (SDR).

9. The method of claim 8, wherein the physiological marker comprises Mrpp2.

10. The method of claim 1, wherein the physiological marker comprises RNAse P complex.

11. The method of claim 1, wherein the physiological marker comprises OXPHOS protein.

12. The method of claim 1, wherein the physiological marker comprises fatty acid oxidation (FAO) protein.

13. The method of claim 1, wherein the therapeutically effective amount comprises 400 mg/kg body weight of the subject to 500 mg/kg body weight of the subject.

14. A method of treating cardiac dysfunction in a subject in need thereof, comprising:
diagnosing cardiac hypertrophy dysfunction in the subject; and
parenterally administering a therapeutically effective amount of a nicotinamide adenine dinucleotide (NAD) precursor to the subject effective to stimulate intracellular oxidized nicotinamide adenine dinucleotide (NAD⁺) synthesis, thereby treating cardiac dysfunction in the subject in need thereof.

15. The method of claim 14, wherein the NAD precursor is nicotinamide riboside chloride (NR).

16. The method of claim 14, wherein the method further comprises treating ventricular cavity dilation in the subject.

17. The method of claim 14, wherein the method further comprises treating lung edema in the subject.

18. The method of claim 14, wherein the therapeutically effective amount comprises 400 mg/kg body weight of the subject to 500 mg/kg body weight of the subject.
